(19) United States
(12) Patent Application Publication
(10) Pub. No.: US 2010/0267704 A1
(43) Pub. Date: Oct. 21, 2010

(54) Autophagy Inducing Compounds and Uses Thereof in Treating Autophagy Associated Diseases

(75) Inventors: Junying Yuan, Waban, MA (US); Dawei Ma, Shanghai (CN); Lihong Zhang, Shanghai (CN)

Correspondence Address: FOLEY HOAG, LLP (w/HUV HMV);PATENT GROUP, 155 SEAPORT BLVD., BOSTON, MA, 02210-2600, US

(73) Assignees: President an Fellows of Harvard Colege, Cambridge, MA (US); Shangha Institute of Organic Chemistry Chineesse Academy of Sciences, Shanghai (CN)

(21) Appl. No.: 12/682,468

(22) PCT Filed: Oct. 10, 2008
(86) PCT No.: PCT/US08/79628

§ 371 Date: Jun. 16, 2010

(30) Foreign Application Priority Data
Oct. 12, 2007 (CN) 200710046992.1

(51) Int. Cl. A61K 31/541; C07D 211/06; A61K 31/445; C07D 307/77; A61K 31/343; C07D 401/12; A61K 31/4545; C07D 401/04; A61K 31/454; C07D 213/80; A61K 31/44; C07D 491/16; A61K 31/407; C07D 487/10; A61K 31/438; C07D 417/06; C12N 5/02; A61P 25/28; A61P 25/16; A61P 35/00

(52) U.S. Cl. 514/225.8; 546/217; 514/315; 549/468; 514/469; 546/194; 514/318; 546/199; 514/322; 546/321; 514/356; 548/417; 514/410; 546/16; 514/278; 544/43; 435/375

(57) ABSTRACT

This invention pertains to a class of autophagy inducing compounds that treat diseases caused by misfolded protein aggregates and a screening method for identifying these compounds.

### RELATED APPLICATIONS

This application claims priority to Chinese Application No. 200710046992.1, filed on Oct. 12, 2007, the entire contents of which are hereby incorporated herein by reference.

TECHNICAL FIELD

This invention pertains to autophagy inducing compounds that are useful in treating or preventing autophagy associated diseases, e.g., diseases caused by misfolded protein aggregates, and to screening methods for identifying these compounds.

### BACKGROUND OF THE INVENTION

Autophagy is a lysosome-dependent process whereby proteins or damaged organelles within a cell are degraded (Klionsky, D. J., and Emr, S. D. (2000). Science. 290: 1717-21). During this process, an autophagosome having a double membrane encloses the component of the cell to be degraded, the autophagosome then fuses with a lysosome which carries out the function of degradation, which results in the recycling of amino acids. This system of degradation and recycling has been conserved to a high degree by evolution and is of key importance in development, growth, aging, illness, death, and other biological processes. Together, the autophagy-lysosome pathway and the ubiquitin-proteasome pathway form the two principal degradation systems of eukaryotic cells; however, the two pathways have different functions in the cell. Autophagy is primarily involved in the degradation of long-lived proteins, protein aggregates, and cellular organelles and other cellular components. Thus, in addition to taking part in various important physiological processes, autophagy appears to have great significance for the treatment of various diseases caused by misfolded protein aggregates in specific tissues and cells.

Under ordinary circumstances, autophagy is maintained at a very low basal level within a cell, but when confronted with starvation or other stress conditions, the level of autophagy is rapidly up-regulated. Seventeen autophagy-related genes (abbreviated “ATG” genes) have already been confirmed through the analysis of yeast genes (Klionsky, D. J., Cregg, J. M., Dunn, W. A., et al. (2003) Dev Cell. 5: 539-45). The proteins encoded by these seventeen genes can be divided into four types, comprising several kinds of serine-threonine kinase (Atg1, Atg13, Atg17) that are involved in regulating upstream autophagy signals (such as mTOR); proteins that are involved in regulating lipase/kinase signal compounds during the initiation stage of the autophagy process (Atg6, Atg14, Vps34, and Vps15); two types of new ubiquitin-like conjugation systems (the Atg8 and Atg12 systems) that are involved in autophagosome formation; and proteins (Atg2, Atg9, and Atg18) that assist ATG molecules bound to an autophagosome during the autophagosome formation process to dissociate from the mature autophagosome. The vast majority of ATG genes in yeast have homologues in high-level eukaryotic biological cells (Mizushima, N. and Klionsky, D. J. (2007) Annu Rev Nutr. 27: 19-40). For example, in mammalian cells, the inhibition of autophagy under non-starvation conditions is mediated by target of rapamycin (mTOR) kinase (Lum, J. J., DeBerardinis, R. J., and Thompson, C. B. (2005) Nat Rev Mol Cell Biol. 6: 439-48). By contrast, C3 P13 kinase, the homologue of yeast Vps34 protein in mammalian cells, is required for the initiation of autophagy.

Autophagy can be induced by many factors both from within and outside the cell, including starvation, nutrient deprivation, bacterial infection, damage to cellular organelles, and protein mismatching. At present, only the mechanism underlying starvation-induced autophagy is understood with relative clarity. However, at the same time, it has been demonstrated that a number of intracellular signaling molecules, such as AMPK, mTOR, C3PI3K, and MAPK, are also involved in autophagy regulation.

### SUMMARY OF THE INVENTION

The present invention addresses the need for further understanding the mechanism of action underlying autophagy and identifying additional small molecular compounds that induce autophagy. The screening methods of the present invention exploit, for example, the localization of LC3-GFP on the membrane of an autophagosome during autophagy and/or fluorescence tagged FYVE domains to detect the level and location of PI(3)P within the cell. LC3, the primate cell homologue of yeast ATG8, is a characteristic protein marker for autophagosomes.

Also disclosed herein are autophagy inducing compounds identified by the screening methods of the present invention, which include seven compounds that have already been approved by the FDA and one compound with known Ca2+ channel activity. These compounds can promote the degradation of long-lived proteins within the cell and reduce over-expression levels of polyQ in transfected cells. These compounds are useful for the treatment of autophagy associated disorders, such as diseases caused by misfolded protein aggregates, e.g., neurodegenerative diseases.

In one aspect, the present invention features a pharmaceutical composition comprising an autophagy inducing compound in an amount effective for treating an autophagy associated disease, wherein said compound is selected from the group including Loperamide, Amiodarone, Niguldipine, Pimozide, Nicardipine, Penitrem A, Fluspirilene, and Trifluoperazine. In one embodiment of this aspect, the pharmaceutical further includes a pharmaceutically acceptable carrier. In another embodiment of this aspect, the autophagy associated disease is a disease caused by misfolded protein aggregates.

In another aspect, the present invention features a pharmaceutical composition comprising an autophagy inducing compound in an amount effective for treating an autophagy associated disease, wherein the compound is at least one compound selected from the group including:
(a) compounds of formula (I):

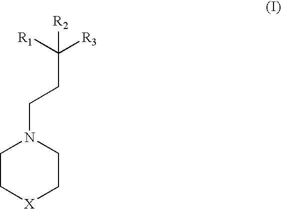

wherein X is selected from CR₄R₅ and NR₆;
R₁ is selected from hydrogen, C₁₋₆ alkyl and phenyl, wherein the alkyl and phenyl are substituted with 0 or 1 halogen;
R₂ is selected from hydrogen, C₁₋₆ alkyl and phenyl, wherein the alkyl and phenyl are substituted with 0 or 1 halogen;
R₃ is selected from

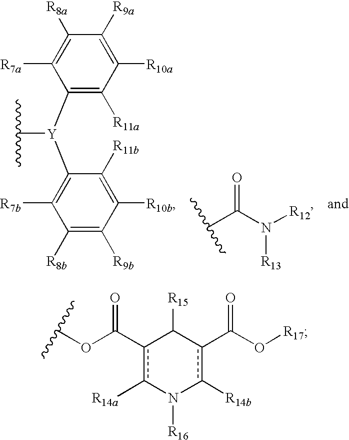

R₄ is selected from hydrogen, hydroxyl, C₁₋₆ alkyl and phenyl;
R₅ is selected from C₁₋₆ alkyl and phenyl, halophenyl, benzimidazole, dihydrobenzimidazole, benzimidazolone;
optionally R₄ and R₅ are taken together to form a 5 or 6 membered heterocycloalkyl comprising two nitrogen atoms, wherein the heterocycloalkyl is substituted with 1, 2 or 3 substituents selected from the group consisting of C₁₋₆ alkyl, phenyl, and ═O;
R₆ is selected from hydrogen and C₁₋₆ alkyl;
R₇ₐ, R₈ₐ, R₉ₐ, R₁₀ₐ, R₁₁ₐ, R_{7b}, R_{8b}, R_{9b}, R_{10b}, and _{11b} are each independently selected from hydrogen, hydroxyl, halogen and C₁₋₆ haloalkyl; optionally R₁₁ₐ and R_{11b} are taken together to form a heterocycle of the following structure:

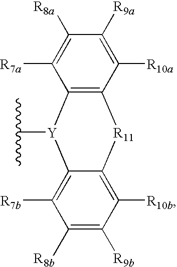

wherein R₁₁ is selected from CH₂, NH, O and S;
R₁₂ and R₁₃ are each independently selected from hydrogen and C₁₋₆ alkyl;
R₁₄ₐ and R_{14b} are each independently selected from hydrogen and C₁₋₆ alkyl;
   R₁₅ is selected from phenyl substituted with 0 or 1 halogen or nitro;
   R₁₆ is selected from hydrogen and C₁₋₆ alkyl;
   Y is N or CH;

and pharmaceutically acceptable salts thereof;
(b) compounds of formula (II):

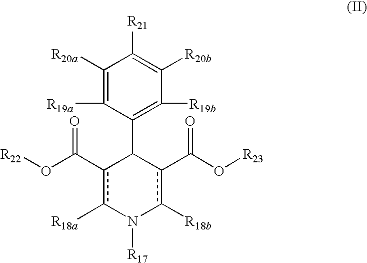

wherein R₁₇ is selected from hydrogen and C₁₋₆ alkyl;
R₁₈ₐ and R_{18b} are each independently selected from hydrogen and C₁₋₆ alkyl;
R₁₉ₐ, R_{19b}, R₂₀ₐ, R_{20b}, and R₂₁ are each independently selected from hydrogen, halogen and nitro;
R₂₂ is selected from hydrogen and C₁₋₆ alkyl;
R₂₃ is selected from —(CH₂)ₙNR₂₄ₐR_{24b} and —(CH₂)ₙOR₂₄ₐ;
R₂₄ₐ and R_{24b} are each independently selected from C₁₋₆ alkyl and phenyl, wherein the alkyl is substituted with 0 or 1 phenyl substituents;
optionally R₂₄ₐ and R_{24b} are taken together with the nitrogen to which they are attached to form a piperidine which is substituted with 0, 1 or 2 phenyl substituents;
n is a positive integer from 2 to 4;

and pharmaceutically acceptable salts thereof.
(c) compounds of formula (III):

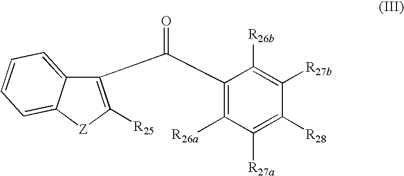

wherein R₂₅ is selected from hydrogen and C₁₋₆ alkyl;
R₂₆ₐ, R_{26b}, R₂₇ₐ, and R_{27b} are each independently selected from hydrogen, halogen and C₁₋₆ alkyl;
R₂₈ is selected from —O(CH₂)ₘNR₂₉ₐR_{29b} and —NH(CH₂)ₘNR₂₉ₐR_{29b};
R₂₉ₐ and R_{29b} are each independently selected from hydrogen and C₁₋₆ alkyl;
Z is O, S or NH;
m is a positive integer from 1 to 3;

and pharmaceutically acceptable salts thereof.
(d) compounds of formula (IV):

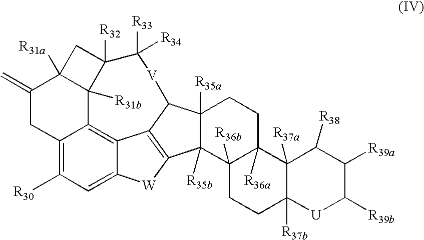

wherein R₃₀ is selected from hydrogen, C₁₋₆ alkyl and halogen;
R₃₁ₐ and R_{31b} are each independently selected from hydrogen, hydroxyl and C₁₋₆ alkyl;
R₃₂ is selected from hydrogen, hydroxyl and C₁₋₆ alkyl;
R₃₃ and R₃₄ are each independently selected from hydrogen and C₁₋₆ alkyl;
R₃₅ₐ and R_{35b} are each independently selected from hydrogen, hydroxyl and C₁₋₆ alkyl;
R₃₆ₐ and R_{36b} are each independently selected from hydrogen, hydroxyl and C₁₋₆ alkyl;
R₃₇ₐ and R_{37b} are each independently selected from hydrogen, hydroxyl and C₁₋₆ alkyl;
R₃₈ is selected from hydrogen, hydroxyl and C₁₋₆ alkyl;
optionally R₃₇ₐ and R₃₈ are taken together to form a three membered heterocycle of the formula:

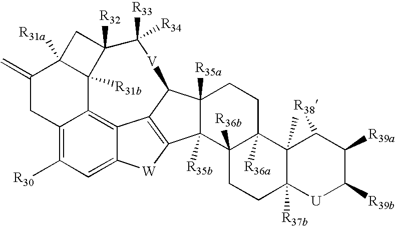

wherein R₃₈′ is O, S or NH;
R₃₉ₐ is selected from hydrogen, hydroxyl and C₁₋₆ alkyl;
R_{39b} is selected from hydrogen, hydroxyl, C₁₋₆ alkyl and C₂₋₆ alkenyl;
U, V and W are each independently selected from O, S, and NH;

and pharmaceutically acceptable salts thereof.

In one embodiment of this aspect, the composition further comprises a pharmaceutically acceptable carrier. In another embodiment of this aspect, the autophagy associated disease is a disease caused by misfolded protein aggregates.

In one aspect, the present invention discloses a method of inducing autophagy in a cell, the method comprising contacting the cell with an autophagy inducing compound in an amount effective to induce autophagy in the cell. In one embodiment of this aspect, the autophagy inducing compound is selected from the group consisting of Loperamide, Amiodarone, Niguldipine, Pimozide, Nicardipine, Penitrem A, Fluspirilene, and Trifluoerazine and pharmaceutically acceptable salts thereof. In another embodiment of this aspect, the compound is at least one compound of formulae (I), (II), (III) or (IV) and pharmaceutically acceptable salts thereof.

In one embodiment of this aspect, the cell is present in a subject. In another embodiment of this aspect, the cell is present in an in vitro cell culture. In a further embodiment of this aspect, the cell is contacted with an autophagy inducing compound at a concentration of about 0.1 μM to about 15.0 μM. In yet another embodiment of this aspect, the cell is contacted with an autophagy inducing compound at a concentration of about 3.0 μM to about 9.0 μM. In a preferred embodiment of this aspect, the cell is selected from the group consisting of neural cells, glial cells, such as astrocytes, oligodendrocytes, ependymal cells, Schwann cells, lymphatic cells, epithelial cells, endothelial cells, lymphocytes, cancer cells, and haematopoietic cells.

In another aspect, the present invention features a method of treating an autophagy associated disease in a subject; the method includes administering to the subject an autophagy inducing compound in an amount effective to treat the disease, thereby treating the disease in the subject. In one embodiment of this aspect, the autophagy associate disease is a disease caused by misfolded protein aggregates. In another embodiment of this aspect, the disease caused by misfolded protein aggregates is selected from the group including: Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, and neuronal intranuclear hyaline inclusion disease. In a further embodiment of this aspect, the disease associated with misfolded protein aggregates is a chronic disease. In yet another embodiment of this aspect, the autophagy associated disease is cancer. In a preferred embodiment of this aspect, the autophagy inducing compound is selected from the group comprising Loperamide, Amiodarone, Niguldipine, Pimozide, Nicardipine, Penitrem A, Fluspirilene, Trifluoerazine, and pharmaceutically acceptable salts thereof. In yet another embodiment of this aspect, the compound is at least one compound of formulae (I), (II), (III) or (IV) and pharmaceutically acceptable salts thereof.

In yet another embodiment of this aspect, the autophagy inducing compound is administered at a concentration of about 0.1 μM to about 15.0 μM. In a preferred embodiment of this aspect, the autophagy inducing compound is administered at a concentration of about 3.0 μM to about 9.0 μM.

In another aspect, the present invention features a kit which includes: (i) a pharmaceutical composition comprising an autophagy inducing compound and (ii) instructions for administering the composition to a subject for the treatment of an autophagy associated disease.

The present invention further provides a method for identifying an autophagy inducing compound, said method includes the steps:

(a) contacting a cell expressing LC3 operatively linked to a detectable tag with a test compound;

(b) determining whether the test compound causes an increase in the expression or intensity of the detectable tag operatively linked to LC3 as compared to a control;

(c) contacting a cell expressing FYVE operatively linked to a detectable tag with the compound identified in step (b) as causing an increase in the expression or intensity of the detectable tag operatively linked to LC3;

(d) determining whether the compound causes a reduction in the expression or intensity of the detectable tag operatively linked to FYVE as compared to a control,

thereby identifying said compound from step (d) which does not cause a reduction in the expression or intensity of the detectable tag operatively linked to FYVE as compared to a control, as an autophagy inducing compound.

In one embodiment of this aspect, the cell expressing LC3 operatively linked to a detectable tag is stably transfected with a construct comprising LC3 operatively linked to a detectable tag. In another embodiment of this aspect, the cell expressing FYVE operatively linked to a detectable tag is stably transfected with a construct comprising FYVE operatively linked to a detectable label. A further embodiment of this aspect includes verifying the identified autophagy inducing compound by testing for an increase in the LC3 II/LC3 I ratio in a cell. Yet another embodiment of this aspect includes

(a) verifying the identified autophagy inducing compound by contacting a cell transfected with GFP-polyglutamine (poly Q)-HA with the identified compound;

(b) determining whether said identified compound induces poly Q degradation; and

(c) selecting an identified compound that induces poly Q degradation.

In one embodiment, the induction of poly Q degradation is tested using immunoblot analysis. Another embodiment of this aspect includes determining whether said test compound is cytotoxic to said cell. In a preferred embodiment of this aspect, the detectable tag operatively linked to LC3 is GFP. In another preferred embodiment of this aspect, the detectable tag operatively linked to FYVE is RFP. In a further embodiment of this aspect, the detectable tag operatively linked to LC3 is an epitope tag and the detectable tag operatively linked to FYVE is an epitope tag. In another embodiment, the epitope tag is selected from the group including HA, V5, HIS, and FLAG. In yet another embodiment, the epitope tag is detected indirectly via the binding of fluorescent conjugated antibodies. In one embodiment, the detectable tag is detected via a method selected from the group including immunoblot analysis, immunohiostochemistry, fluorescence micrsocopy, and indirect immunofluoresence. In a further embodiment of this aspect, the cell expressing LC3 operatively linked to a detectable tag is selected from the group consisting of neural cells, glial cells, such as astrocytes, oligodendrocytes, ependymal cells, Schwann cells, lymphatic cells, epithelial cells, endothelial cells, lymphocytes, cancer cells, and haematopoietic cells. In yet another embodiment of this aspect, the cell expressing FYVE operatively linked to a detectable tag is selected from the group consisting of neural cells, glial cells, such as astrocytes, oligodendrocytes, ependymal cells, Schwann cells, lymphatic cells, epithelial cells, endothelial cells, lymphocytes, cancer cells, and haematopoietic cells.

Other features and advantages of the invention will be apparent from the following Detailed Description, the Drawings, and the Claims.

### BRIEF DESCRIPTION OF THE DRAWINGS

FIG. 1: Depicts the percent increase of endogenous LC3 II induced by the eight authophagy inducing compounds identified herein. The abbreviations used in FIG. 1 are as follows: D: DMSO; R: rapamycin; 3: amiodarone; 4: niguldipine; 5: trifluoperazine; 6: loperamide; 7: penitrem A; 8: pimozide; 9: fluspirilene; 10: nicardipine.

FIG. 2: Depicts the increased polyQ degradation induced by the eight compounds identified herein. From left to right: first column: top: DMSO; bottom: sample treated with rapamycin. Second through ninth columns: samples treated with fluspirilene, pimozide, trifluoperazine, amiodarone, loperamide, nicardipine, niguldipine, and penitrem A, respectively. From top to bottom, samples were treated with specified dilutions of the eight compounds. Sequentially, samples were diluted with the compounds at the concentration used in the screening (for numbers, see FIG. 1), and then the samples were treated with compounds diluted in the ratio 1:2.5; 1:5; and 1:10.

### DETAILED DESCRIPTION OF THE INVENTION

### A. Definitions

In order to more clearly and concisely describe the subject matter of the claims, the following definitions are intended to provide guidance as to the meaning of specific terms used herein.

It is to be noted that the singular forms “a,” “an,” and “the” as used herein include “at least one” and “one or more” unless stated otherwise. Thus, for example, reference to “a pharmacologically acceptable carrier” includes mixtures of two or more carriers as well as a single carrier, and the like.

Numerous values and ranges are recited in connection with various embodiments of the present invention, e.g., amount of a compound of formula (I) or (II) present in a composition. It is to be understood that all values and ranges which fall between the values and ranges listed are intended to be encompassed by the present invention unless explicitly stated otherwise. The term “about” as used herein in association with parameters, ranges and amounts, means that the parameter or amount is within ±1% of the stated parameter or amount.

The term “autophagy” refers to the catabolic process involving the degradation of a cell's own components; such as, long lived proteins, protein aggregates, cellular organelles, cell membranes, organelle membranes, and other cellular components. The mechanism of autophagy may include: (i) the formation of a membrane around a targeted region of the cell, separating the contents from the rest of the cytoplasm, (ii) the fusion of the resultant vesicle with a lysosome and the subsequent degradation of the vesicle contents. For example, the term autophagy may refer to one of the mechanisms by which a starving cell re-allocates nutrients from unnecessary processes to more essential processes. Also, for example, autophagy may inhibit the progression of some diseases and play a protective role against infection by intracellular pathogens.

The term “autophagy inducing compound” refers to a compound that induces autophagy in a cell. The term autophagy inducing compound, as used herein, comprises the specific compounds disclosed herein. For example, the term autophagy inducing compound comprises; Loperamide, Amiodarone, Niguldipine, Pimozide, Nicardipine, Penitrem A, Fluspirilene, and Trifluoerazine; as well as pharmaceutical acceptable salts thereof and metabolites or variants thereof as described herein.

The term “screening method” refers to a method of investigating a large number of elements. For example the term screening method may refer to a method of investigating of a large number of compounds for one or more properties.

The term “cell culture” refers to the process by which prokaryotic, or eukaryotic cells are grown under controlled conditions. For example, the term “cell culture” may refer to the culturing of cells derived from multicellular eukaryotes, preferably mammalian cells.

The term “detectable tag” refers to any moiety that can be detected by a skilled practitioner using art known techniques. Detectable tags for use in the screening methods of the present invention may be peptide sequences. Optionally the detectable tag may be removable by chemical agents or by enzymatic means, such as proteolysis. For example the term “detectable tag” includes chitin binding protein (CBP)-tag, maltose binding protein (MBP)-tag, glutathione-S-transferase (GST)-tag, poly(His)-tag, FLAG tag, Epitope tags, such as, V5-tag, c-myc-tag, and HA-tag, and fluorescence tags such as green fluorescent protein (GFP), red fluorescent protein (RFP), yellow fluorescent protein (YFP), blue fluoresecnt protein (BFP), and cyan fluoresecent protein (CFP); as well as derivatives of these tags. The term “detectable tag” also includes the term “detectable marker”.

The term “LC3” refers to microtubule-associated protein light chain 3 (LC3). LC3 is positioned on the pre-autophagosome and on the surface of the autophagosome membrane, and is widely used as an autophagosome membrane marker. LC3 includes all forms of LC3 including LC3 I or LC3 II. The term “LC3 I” refers to cytosolic LC3, whereas the term “LC3 II” refers to membrane bound LC3. LC3 II is present both inside and outside autophagosomes.

The term “FYVE domain” refers to the FYVE zinc finger domain of a protein which binds two zinc ions. The FYVE domain has eight potential zinc coordinating cysteine positions. FYVE domains are know in the art to bind Phosphatidylinositol 3-phosphate. FYVE domains are described in, for example, Stenmark, H., Aasland, R., and Driscoll, P. C. (2002) FEBS Lett. 513: 77-84 the entire contents of which (as they relate to, for example, FYVE domains and methods of making and using the same) are incorporated herein by reference.

The term “fluorescent conjugated antibody” refers to a primary or secondary antibody conjugated to a fluorescent probe or fluorophore. The fluorescent conjugated antibody may be used as a detectable probe.

The term “detectable probe”, includes any molecule that specifically binds to a nucleic acid sequence or to a protein that is being monitored, and which can be labeled so that the required targets can be detected. For example, the probe may be radiolabeled or chemically tagged. In another example, specific monoclonal antibodies may be used to detect proteins, and the monoclonal antibody can be labeled so that the protein of interest can be detected.

As used herein, the term “operatively linked”, is intended to have its ordinary meaning known in the art. For example, it is intended to mean that the nucleotide sequence that codes for the protein of interest is linked to the nucleotide sequence that codes for the detectable tag in a manner which allows for expression of the operatively linked protein sequence (e.g., in an in vitro transcription/translation system or in a host cell when the vector is introduced into the host cell).

The term “misfolded protein aggregates” refers to a mass of misfolded proteins, wherein said proteins have not adopted the appropriate three-dimensional structure, i.e., tertiary structure. For example, the misfolded proteins may have clustered together to form an assemblage of misfolded proteins.

The term “autophagy associated disease” includes a disease that can be treated by the induction of autophagy. Examples of such diseases include diseases caused by misfolded protein aggregates. The term “disease caused by misfolded protein aggregates” is intended to include any disease, disorder or condition associated with or caused by misfolded protein aggregates. For example, such diseases include Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, and neuronal intranuclear hyaline inclusion disease. The term “autophagy associated disease” also includes cancer e.g., any cancer wherein the induction of autophagy would inhibit cell growth and division, reduce mutagenesis, remove mitochondria and other organelles damaged by reactive oxygen species or kill developing tumor cells. Autophagy associated diseases can be chronic diseases.

The term “chronic disease” refers to a persistent and lasting disease or medical condition, or one that has developed slowly.

The term “effective” amount refers to the amount of an autophagy inducing compound of the present invention required to treat or prevent an autophagy associated disease, e.g., a disease associated with misfolded protein aggregates. The effective amount of an autophagy inducing compound of the invention used to practice the invention for therapeutic or prophylactic treatment of autophagy associated diseases varies depending upon the manner of administration, the age, body weight, and general health of the subject. An effective amount of an autophagy inducing compound, as defined herein may vary according to factors such as the disease state, age, and weight of the subject, and the ability of the autophagy inducing compound to elicit a desired response in the subject. Dosage regimens may be adjusted to provide the optimum therapeutic response. An effective amount is also one in which any toxic or detrimental effects (e.g., side effects) of the autophagy inducing compound are outweighed by the therapeutically beneficial effects. A therapeutically effective amount of an autophagy inducing compound (i.e., an effective dosage) may range from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an autophagy inducing compound can include a single treatment or, preferably, can include a series of treatments. In one example, a subject is treated with an autophagy inducing compound in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of an autophagy inducing compound used for treatment may increase or decrease over the course of a particular treatment.

The term “pharmaceutical composition” refers to a composition containing an autophagy inducing compound of the invention formulated with one or more pharmaceutical-grade excipients in a manner that conforms with the requirements of a governmental agency regulating the manufacture and sale of pharmaceuticals as part of a therapeutic regimen for the treatment or prevention of disease in a mammal (e.g., manufactured according to GMP regulations and suitable for administration to a human). Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or any other formulation described herein.

The term “pharmaceutically acceptable carrier” refers to any such carriers known to those skilled in the art to be suitable for the particular mode of administration. For example, the term “pharmaceutically acceptable carrier” includes any and all solvents, dispersion media, coatings, antibacterial and antifungal agents, isotonic and absorption delaying agents and the like, that may be used as a media for a pharmaceutically acceptable substance. In addition, the active materials can also be mixed with other active materials that do not impair the desired action, or with materials that supplement the desired action, or have another action. The autophagy inducing compounds may be formulated as the sole pharmaceutically active ingredient in the composition or may be combined with other active ingredients.

As used herein, the term “treating” refers to administering a pharmaceutical composition for prophylactic and/or therapeutic purposes. To “prevent disease” refers to prophylactic treatment of a subject who is not yet ill, but who is susceptible to, or otherwise at risk of, a particular disease. To “treat disease” or use for “therapeutic treatment” refers to administering treatment to a subject already suffering from a disease to improve or stabilize the subject's condition. Thus, in the claims and embodiments, treating is the administration to a subject either for therapeutic or prophylactic purposes.

The term “subject” includes humans, and non-human animals amenable to therapy, e.g., preferably mammals and animals susceptible to an autophagy associated disease, such as a disease associated with misfolded protein aggregates, including non-human primates, transgenic animals, mice, rats, dogs, cats, rabbits, pigs, chickens, sheep, horses, and cows. Preferably, the subject is a human subject.

As used herein, “alkyl” groups include saturated hydrocarbons having one or more carbon atoms, including straight-chain alkyl groups (e.g., methyl, ethyl, propyl, butyl, pentyl, hexyl, etc.), cyclic alkyl groups (or “cycloalkyl” or “alicyclic” or “carbocyclic” groups) (e.g., cyclopropyl, cyclopentyl, cyclohexyl, etc.), branched-chain alkyl groups (isopropyl, tert-butyl, sec-butyl, isobutyl, etc.), and alkyl-substituted alkyl groups (e.g., alkyl-substituted cycloalkyl groups and cycloalkyl-substituted alkyl groups). The term “C₁-₆” as in “C₁-₆ alkyl” means alkyl groups containing 1 to 6 carbon atoms. The terms “alkenyl” and “alkynyl” refer to unsaturated aliphatic groups analogous to alkyls, but which contain at least one double or triple carbon-carbon bond respectively.

The term “heterocycle” includes closed ring structures in which one or more of the carbon atoms in the ring is an element other than carbon, for example, nitrogen, sulfur, or oxygen. Heterocyclic groups may be saturated or unsaturated. Additionally, heterocyclic groups (such as pyrrolyl, pyridyl, isoquinolyl, quinolyl, purinyl, and furyl) may have aromatic character, in which case they may be referred to as “heteroaryl” or “heteroaromatic” groups. Exemplary heterocyclic groups include, but are not limited to

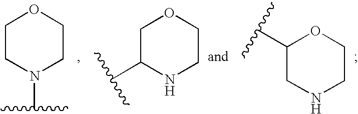

heterocycloalkyls such as morpholinyl, e.g.,

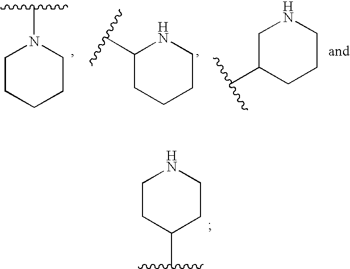

piperidinyl, e.g.,

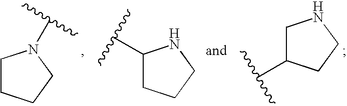

pyrrolidinyl, e.g.,

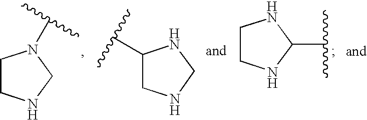

imidazolidinyl, e.g.,

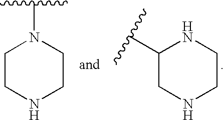

piperazinyl, e.g.,

When specified, the chemical moieties of the compounds of formula (I) or (II), including those groups discussed above, may be “substituted or unsubstituted.” In some embodiments, the term “substituted” means that the moiety has substituents placed on the moiety other than hydrogen (i.e., in most cases, replacing a hydrogen), which allow the molecule to perform its intended function. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is meant to include all permissible substituents of organic compounds. In a broad aspect, permissible substituents include acyclic and cyclic, branched and unbranched, carbocyclic and heterocyclic, aromatic and nonaromatic substituents of organic compounds. The compounds of formula (I) or (II) may have one or more substitutions, as described herein.

As used in the description and drawings herein, an optional single/double bond is represented by a solid line together with a dashed line, and refers to a covalent linkage between two carbon atoms which can be either a single bond or a double bond. For example, the structure:

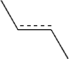

can represent either butane or butene.

As used herein, the notation

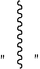

in any substituent structure refers to the point at which the substituent is linked to the core molecule.

When compounded chemical names, e.g., “alkylaryl,” “aryloxy,” and the like, are used herein, they are understood to have a specific connectivity to the core of the chemical structure. The moiety listed farthest to the right (e.g., aryl in “alkylaryl”), is the moiety which is directly connected to the core. That is, for example, in a structure V—CH₂CH₂CH₃, when the variable “V” is an alkylaryl, the structure is understood to be alkyl-aryl-CH₂CH₂CH₃.

As used herein, the term “compound” is intended to mean a substance made up of molecules that further consist of atoms. A compound generally refers to a chemical entity, whether in the solid, liquid or gaseous phase, and whether in a crude mixture or purified and isolated. Compounds encompass the chemical compound itself as well as, where applicable: amorphous and crystalline forms of the compound, including polymorphic forms, said forms in mixture or in isolation; free acid and free base forms of the compound; isomers of the compound, including geometric isomers, optical isomers, and tautomeric isomers, said optical isomers to include enantiomers and diastereomers, chiral isomers and non-chiral isomers, said optical isomers to include isolated optical isomers or mixtures of optical isomers including racemic and non-racemic mixtures; said geometric isomers to include transoid and cisoid forms, where an isomer may be in isolated form or in admixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, including acid addition salts and base addition salts, including organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different.

The term “test compound” includes any chemical composition or drug to be tested, screened or selected using the screening methods of the present invention. For example, the term test compound includes any chemical composition or drug that may induce autophagy in a cell.

### B. PI 3-Kinases and Autophagy

Class III phosphoinositide 3-kinases (PI 3-kinases or PI3K′s) catalyze the phosphorylation of phosphatidylinositol (PI), generating phosphatidylinositol-3-phosphate (PtdIns(3)P or PI(3)P). The latter is of key importance in the endocytic and autophagosome membrane transport processes (Simonsen, A., Wurmser, A. E., Emr, S. D., et al. (2001) Curr. Opin. Cell Biol. 13: 485-92). At the same time, the compounds formed by Vps34/beclin1 (Vps34 and beclin1 are the respective homologues in mammalian cells of yeast class III PI3K′s and ATG6) are involved in regulating the signal for autophagy initiation (Nobukuni, T., Kozma, S. C., and Thomas, G. (2007) Curr Opin Cell Biol. 19: 135-41). Therefore, PI(3)P levels should not decrease to a significant extent during autophagy.

The FYVE domain, being composed of approximately 70 amino acid residues, forms a zinc finger protein structure, which specifically binds PtdIns(3)P. Generally, PtdIns(3)P collects proteins that contain the FYVE domain and binds to the membrane of an organelle in order to participate in protein transport and other similar processes. Hence, fluorescent-marked FYVE domains are frequently used to detect the level and location of PI(3)P within the cell (Stenmark, H., Aasland, R., and Driscoll, P. C. (2002) FEBS Lett. 513: 77-84). Cell lines stably expressing a FYVE-RFP protein mixture were also used in order to observe the effect of a compound on a FYVE domain and thereby indirectly observe the effect of the compound on autophagy. Compounds with markedly reduced FYVE were eliminated.

### C. Protein Degradation

The process of autophagy is a protein degradation process, which primarily mediates the degradation of cellular organelles and long-lived proteins within the cell. Therefore, the detection of whether a test compound promotes the degradation of long-lived proteins within the cell may be used to determine whether the test compound induces autophagy. In addition, large aggregates of misfolded proteins are a prominent feature of many neurodegenerative diseases. For example, the pathogenic mechanism of Huntington's disease is that a large amount of polyglutamine (polyQ) protein accumulates in the neurons and cannot be cleared away. In this situation, autophagy is regarded as a mechanism for clearing away polyQ (Wullschleger, S., Loewith, R., and Hall, M. N. 2006. Cell. 124:471-84). For example, the autophagy inducing agent rapamycin is a compound frequently used to eliminate polyQ aggregates. Hence, tests of polyQ elimination and degradation are likewise important evidence of the occurrence of autophagy.

### D. Screening Method

The present invention discloses a simple, convenient, and highly effective method for screening compounds that modulate autophagy. In one aspect, the present invention provides a method for identifying autophagy inducing compounds that are useful for treating diseases caused by misfolded protein aggregates and for identifying compounds that modulate (induce or inhibit) the occurrence of autophagy in eukaryotic cells.

In one embodiment, the screening method of the present invention combines autophagic molecular mechanisms with the advanced technology of high through-put screening and thereby establishes a screening method, based on changes in an image, for compounds that modulate autophagy.

The screening methods of the present invention comprise the following elements as discussed in detail herein:

1. Treatment of a cell, e.g., a cell line, that expresses, e.g., stably expresses, LC3-GFP with test compounds (for example, the ICCB known bioactive library, BIOMOL may be used). In one embodiment of this element, dimethyl sulfoxide solvent (DMSO) may be used as a negative control. In another embodiment of this element, a known inducer of autophagy may be used as a positive control. In a preferred embodiment of this element, rapamycin is used as a positive control.

2. High through-put analysis of fluorescence intensity and the number of cells remaining after treatment may be performed by analyzing changes in LC3-GFP fluorescence. Compounds that reduce the number of cells or do not increase LC3-GFP may be eliminated.

In one embodiment, any compound that increased the intensity of LC3-GFP by more than 50% as compared to a control may be selected. For example, any compound that increases the intensity of LC3-GFP fluorescence by more than about 50%, 55%, 60%, 65%, 70%, 75%, 80%, 85%, 90%, or 95% as compared to a control may be selected. It is to be understood that all values and ranges between these values and ranges are meat to be encompassed by the present invention.

In another embodiment, any compound that induces cell death in greater than about 30% of the cell population may be eliminated as a candidate. For example, any compound that induces cell death in greater than about 25%, 25%, 30%, 35%, 40%, 45%, 50%, 60%, 70%, or 80% of the cell population may be eliminated as a candidate. It is to be understood that all values and ranges between these values and ranges are meant to be encompassed by the present invention.

3. Treatment of a cell, e.g., a cell line, that expresses, e.g., stably expresses, FYVE-RFP with the compounds that increased LC3-GFP in the foregoing step. For example, a compound is selected if a 2-fold, 3-fold, 4-fold, 5-fold, 6-fold, 7-fold, 8-fold, 9-fold, 10-fold or higher increase in LC3-GFP is observed. In one embodiment of this element, DMSO solvent may be used as a negative control. In another embodiment of this element, a known inducer of autophagy may be used as a positive control. In a preferred embodiment of this element, rapamycin is used as a positive control.

4. High through-put analysis of changes in FYVE-RFP fluorescence after treatment are then performed. Compounds that reduce FYVE-RFP fluorescence are eliminated. For example, compounds that reduce FYVE-RFP fluorescence by 2-fold, 3-fold, 4-fold, 5-fold, 6-fold, 7-fold, 8-fold, 9-fold, 10-fold or more are eliminated.

5. Each of the remaining compounds that increase LC3-GFP fluorescence and do not reduce FYVE-RFP fluorescence may be selected as autophagy inducing compounds.

6. Further verification of the autophagy inducing compounds identified in steps 5, may be performed through analysis of the LC3 II/LC3 I ratio in a cell (e.g., the same cell as in Step 1 or a different cell) and/or tests of long-lived protein degradation and polyQ degradation.

When a compound registers markedly more strongly than a control, e.g., dimethyl sulfoxide, on all three of the foregoing indexes, it is identified as an autophagy inducing compound.

In the method of the invention for screening autophagy inducing compounds, the effect of a potential autophagy inducing compound may be assayed by testing the level of expression of LC3 and PI(3)P in a cell using techniques well established in the art. For example, fluorescent conjugated FYVE is often used to detect the level and location of PI(3)P in the cell. In a preferred embodiment, the expression level of LC3 and /or FYVE may assayed by conjugating the protein to a detectable tag, such as green fluorescent protein or red fluorescent protein. The expression level and or fluorescent intensity of LC3 and/or FYVE can be assayed by fluorescence microscopy.

Additionally or alternatively, the level of expression of LC3, LC3 conjugated to a detectable tag, and FYVE conjugated to a detectable tag may assayed by obtaining a cell sample after treatment with a potential autophagy inducing compound and detecting the level of polypeptide or mRNA. In addition, LC3 and/or FYVE may be conjugated to a detectable tag, e.g., an epitope tag, thus the expression level of LC3 or FYVE in the cell may assayed indirectly by obtaining a cell sample after treatment with a potential autophagy inducing compound and detecting the expression level of the detectable tag conjugated to LC3 or FYVE.

For example, an assay for detecting the levels of mRNA in a sample may be selected from the group including Northern blot, RT-PCR, Quantitative PCR (QPCR), in situ hybridization, and gene expression microarray analysis. An assay for detecting the levels of a polypeptide in a sample may be selected from the group including Western blot, immunohistochemistry, indirect immunofluoresence, fluorescence microscopy, enzyme-linked immunosorbent assay (ELISA), and antibody microarray analysis. In an exemplary embodiment of these aspects, an antibody that binds LC3 polypeptide or the detectable tag may be used to detect expression levels of LC3 polypeptide or the detectable tag conjugated to LC3 or FYVE.

The specific examples described above for detecting the expression levels of mRNA and/or a polypeptide are representative examples and are not intended to be limiting. Other suitable approaches for assaying the expression level of mRNA and/or a polypeptide are known in the art. It will be readily understood by the ordinarily skilled artisan that essentially any technical means established in the art for detecting mRNA and/or polypeptide levels in a sample can be adapted to the detection of LC3, LC3 conjugated to a detectable tag or FYVE conjugated to a detectable tag as discussed herein and applied in the methods of the current invention for selecting an autophagy inducing compound.

The foregoing screening methods may be performed using any suitable cell. For example, neural cells, glial cells, such as astrocytes, oligodendrocytes, and ependymal cells, Schwann cells, lymphatic cells, epithelial cells, endothelial cells, lymphocytes, cancer cells, and haematopoietic cells may be used.

The method of the present invention may be practiced using in vitro cell culture methods and cell lines. For example, cell lines used in the method of the present invention may include: hepatoma cells, neuroglioma cells, glioma cells, cervical cancer cells, glioblastoma cells, breast cancer cells, prostate cancer cells, primary cell lines, embryonic fibroblast cells, kidney cells, melanoma cells, lymphoma cells, colorectal carcinoma cells, osteosarcoma cells, myeloblast cells, colon epithelium cells, T-cell leukemia cell lines, carcinoma cell lines, head and neck carcinoma cells, skin epithelium cells, lung carcinoma cells, bones marrow cells, melanoma cells, neuroblastoma cells, pancreatic-adenocarcinoma cells, mesenchymal cells, ovarian cell, prostatic-adenocarcinoma cells, mammary gland cells, embryonic cells, astrocytoma cell lines, and B-cells.

### E. Autophagy Inducing Compounds

In one aspect, the present invention provides a class of autophagy inducing compounds that may be used to treat or prevent diseases caused by misfolded protein aggregates.

Examples of such compounds are presented below:

| Name of | | |
|---|---|---|
| Compound | Structure | Reference data |
| Fluspirilene | 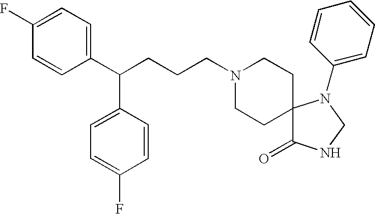 | www.ncbi.nlm.nih.gov/ sites/entrez? cmd = search&db = pccom pound&term = fluspirilene |
| Amiodarone | 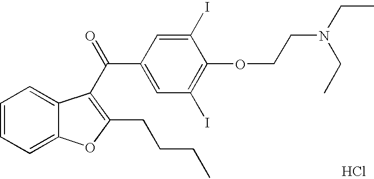 | www.ncbi.nlm.nih.gov/ sites/entrez? cmd = search&db = pccom pound&term = amiodarone |
| Loperamide | 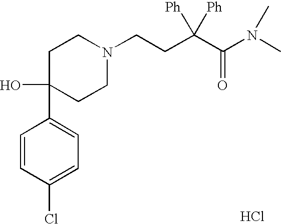 | www.ncbi.nlm.nih.gov/ sites/entrez? db = pccom pound&term = loperamide |
| Nicardipine | 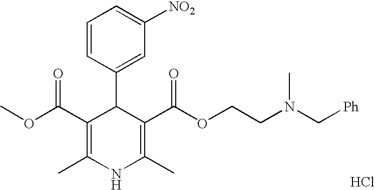 | www.ncbi.nlm.nih.gov/ sites/entrez? cmd = search&db = pccom pound&term = nicardipine |
| Niguldipine | 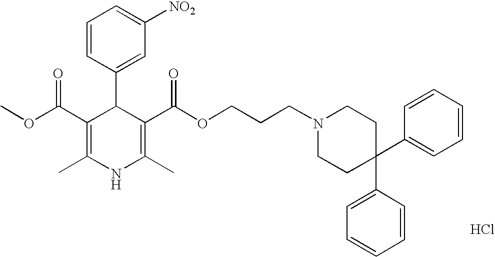 | www.ncbi.nlm.nih.gov/ sites/entrez? cmd = search&db = pccom pound&term = niguldipine |
| Penitrem A | 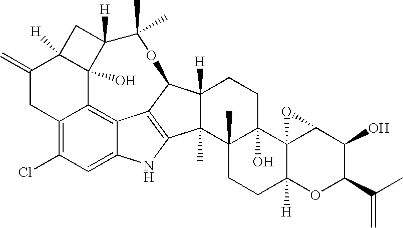 | www.ncbi.nlm.nih.gov/ sites/entrez? cmd = search&db = pccom pound&term = Penitrem % 20 A |
| Pimozide | 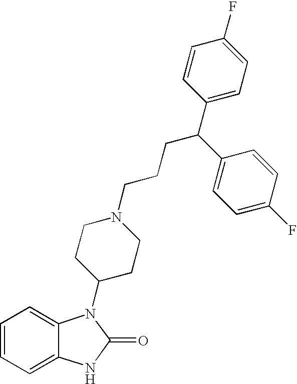 | www.ncbi.nlm.nih.gov/ sites/entrez? cmd = search&db = pccom pound&term = pimozide |
| Trifluoperazine | 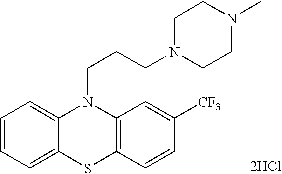 | www.ncbi.nlm.nih.gov/ sites/entrez? cmd = search&db = pccom pound&term = trifluoperazine |

Fluspirilene, an FDA-approved phenothiazine tranquilizer (antipsychotic drug), has been used to treat schizophrenia (Meijer, A. J. and Codogno, P. 2004. Int J Biochem Cell Biol. 36: 2445-62). This molecule may function by blocking adrenaline and dopamine transport in the central nervous system (Janssen, P. A., Niemegeers, C. J., Schellekens, K. H., et al. 1970. Arzneimittelforschung. 20: 1689-98).

Trifluoperazine is another FDA-approved tranquilizer, which, like fluspirilene, can effectively treat acute schizophrenia (Janssen, P. A., Niemegeers, C. J., Schellekens, K. H., et al. 1970. Arzneimittelforschung. 20: 1689-98). In addition, trifluoperazine has also been reported to inhibit calmodulin activity and mitochondrial permeability transition pore (MTP), and to reduce the toxic effect that expanded polyglutamine associated with Huntington's disease has on cells (Stokes, H. B. 1975. Dis Nery Syst. 36: 102-5). In addition, it has also been found that it is a calcium ion channel blocker.

Pimozide is an FDA-approved tranquilizer used in the treatment of chronic schizophrenia. It may act on central aminergic receptors. At high doses, this compound may also affect the degradation of norepinephrine.

Three additional autophagy inducing agents niguldipine, nicardipine, and amiodarone are all FDA-approved drugs for the treatment of cardiovascular disorders, including high blood pressure, angina pectoris, arrhythmia, and the like. Moreover, these compounds have been used to inhibit intracellular Ca2+ current. Among them, niguldipine acts as an inhibitor of Type-T Ca2+ current in cardiac myocytes. As a dihydropyridine-type Ca2+ channel blocker, nicardipine is frequently used to treat chronic angina pectoris, high blood pressure, and Raynaud's phenomenon. Amiodarone is another highly effective anti-arrhythmia drug. It also blocks Ca2+ channels.

Loperamide is a heterocyclic piperidine derivative. As an FDA-approved drug to treat diarrhea, it can effectively improve gastrointestinal symptoms. Loperamide can block high-voltage-activated Ca2+ channels and reactions to N-methyl-D-aspartate in the hippocampal neurons of rabbits and mice (Girotti, F., Carella, F., Scigliano, G., et al. 1984. J Neurol Neurosurg Psychiatry. 47: 848-52). In addition, loperamide can also block voltage-dependant Ca2+ channels in cultured dorsal root ganglions (Church, J. Fletcher, E. J., Abdel-Hamid, K. et al. 1994. Mol Pharmacol. 45: 747-57).

Penitrem A, a fungal neurotoxin discovered in ryegrass, can selectively block Ca2+-activated K+ channels (100% blockage is achieved by 10-nM penitrem A). It has been reported that this compound may possess marked neurotoxicity and can cause severe tremors or ataxia (Hagiwara, K., Nakagawasai, O., Murata, A., et al. 2003. Neurosci Res. 46: 493-7); however the results presented herein show that this drug does not destroy H4 cells.

In some embodiments, the present invention is directed to autophagy inducing compounds of formula (I):

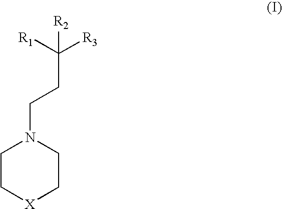

wherein X is selected from CR₄R₅ and NR₆;
R₁ is selected from hydrogen, C₁₋₆ alkyl and phenyl, wherein the alkyl and phenyl are substituted with 0 or 1 halogen;
R₂ is selected from hydrogen, C₁₋₆ alkyl and phenyl, wherein the alkyl and phenyl are substituted with 0 or 1 halogen;
R₃ is selected from

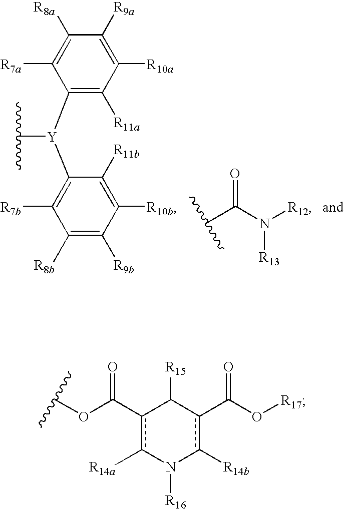

R₄ is selected from hydrogen, hydroxyl, C₁₋₆ alkyl and phenyl;
R₅ is selected from C₁₋₆ alkyl and phenyl, halophenyl, benzimidazole, dihydrobenzimidazole, benzimidazolone;
optionally R₄ and R₅ are taken together to form a 5 or 6 membered heterocycloalkyl comprising two nitrogen atoms, wherein the heterocycloalkyl is substituted with 1, 2 or 3 substituents selected from the group consisting of C₁₋₆ alkyl, phenyl, and ═O;
R₆ is selected from hydrogen and C₁₋₆ alkyl;
R₇ₐ, R₈ₐ, R₉ₐ, R₁₀ₐ, R₁₁ₐ, R_{7b}, R_{8b}, R_{9b}, R_{10b}, and R_{11b} are each independently selected from hydrogen, hydroxyl, halogen and C₁₋₆ haloalkyl; optionally R₁₁ₐ and R_{11b} are taken together to form a heterocycle of the following structure:

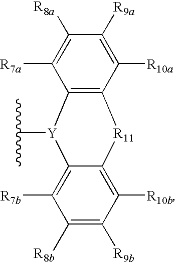

wherein R₁₁ is selected from CH₂, NH, O and S;
R₁₂ and R₁₃ are each independently selected from hydrogen and C₁₋₆ alkyl;
R₁₄ₐ and R_{14b} are each independently selected from hydrogen and C₁₋₆ alkyl;
R₁₅ is selected from phenyl substituted with 0 or 1 halogen or nitro;
R₁₆ is selected from hydrogen and C₁₋₆ alkyl;
Y is N or CH;

and pharmaceutically acceptable salts thereof.

In some embodiments R₁ is phenyl. In some embodiments R₁ is H. In some embodiments R₂ is phenyl. In some embodiments R₂ is H.

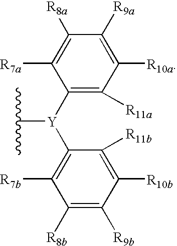

In some embodiments, R₃ is In some embodiments, Y is N. In some embodiments, Y is CH. In some embodiments, R₇ₐ, R₈ₐ, R₉ₐ, R₁₀ₐ, R₁₁ₐ, R_{7b}, R_{8b}, R_{9b}, R_{10b}, and R_{11b} are each independently selected from hydrogen and halogen. In some embodiments, R₇ₐ, R₈ₐ, R₁₀ₐ, R₁₁ₐ, R_{7b}, R_{8b}, R_{10b}, and R_{11b} are each independently hydrogen. In some embodiments, R₉ₐ and R_{9b} are each independently halogen. In some embodiments, R₉ₐ and R_{9b} are each independently fluorine. In some embodiments, R₇ₐ, R₈ₐ, R₉ₐ, R₁₀ₐ, R_{7b}, R_{8b}, R_{9b}, and R_{10b} are each independently selected from hydrogen and C₁₋₆ haloalkyl. In some embodiments, R₇ₐ, R₉ₐ, R₁₀ₐ, R_{7b}, R_{8b}, R_{9b}, and R_{10b} are each independently hydrogen. In some embodiments, R₈ₐ is C₁₋₆ haloalkyl, e.g., trifluoromethyl. In some embodiments, R₁₁ₐ and R_{11b} are taken together to form a heterocycle of the following structure:

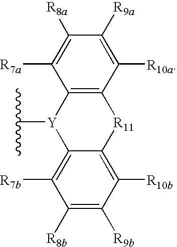

In some embodiments, R₁₁ is S.

In some embodiments, R₃ is

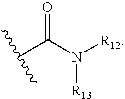

In some embodiments, R₁₂ is C₁₋₆ alkyl, e.g., methyl. In some embodiments, R₁₃ is C₁₋₆ alkyl, e.g., methyl.

In some embodiments, R₃ is

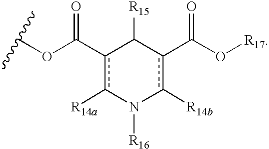

In some embodiments R₁₄ₐ is C₁₋₆ alkyl, e.g., methyl. In some embodiments, R_{14b} is C₁₋₆ alkyl, e.g., methyl. In some embodiments, R₁₅ is phenyl substituted with 1 nitro, e.g., phenyl substituted in the meta position with 1 nitro. In some embodiments, R₁₆ is hydrogen.

In some embodiments, the bonds represented by the structure

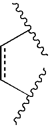

are each independently double bonds.

In some embodiments, R₄ is hydrogen. In some embodiments, R₄ is hydroxyl. In some embodiments, R₄ is phenyl.

In some embodiments, R₅ is phenyl. In some embodiments, R₅ is halophenyl, e.g., chlorophenyl. In some embodiments, R₅ is benzimidazolone.

In some embodiments, R₄ and R₅ are taken together to form a 5 membered heterocycloalkyl comprising two nitrogen atoms, wherein the heterocycloalkyl is substituted with 1 or 2 substituents selected from the group consisting of phenyl and ═O. In some embodiments, R₄ and R₅ are taken together to form an imidazolidinyl group substituted with 1 or 2 substituents selected from the group consisting of phenyl and ═O. In some embodiments, R₄ and R₅ are taken together to form a heterocycle of the following structure:

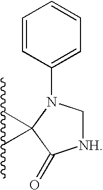

In some embodiments, R₆ is C₁₋₆ alkyl. In some embodiments, R₆ is methyl.

In some embodiments, the pharmaceutically acceptable salt is a hydrochloride salt.

In some embodiments, the autophagy inducing compounds of the present invention include the following compounds:

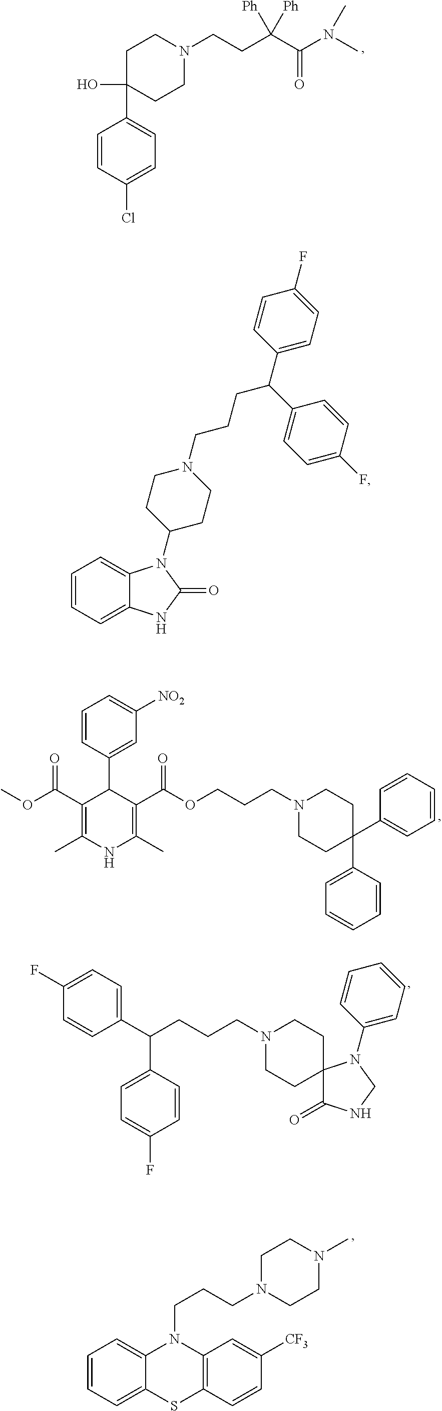

and pharmaceutically acceptable salts thereof.

In some embodiments, the present invention is directed to autophagy inducing compounds of formula (II):

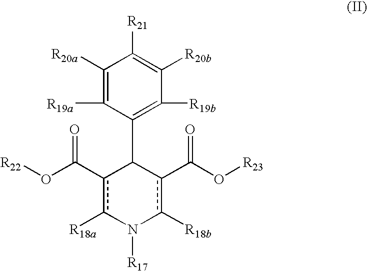

wherein R₁₇ is selected from hydrogen and C₁-₆ alkyl;
R₁₈ₐ and R_{18b} are each independently selected from hydrogen and C₁₋₆ alkyl;
R₁₉ₐ, R_{19b}, R₂₀ₐ, R_{20b}, and R₂₁ are each independently selected from hydrogen, halogen and nitro;
R₂₂ is selected from hydrogen and C₁₋₆ alkyl;
R₂₃ is selected from —(CH₂)ₙNR₂₄ₐR_{24b} and —(CH₂)ₙOR₂₄ₐ;
R₂₄ₐ and R_{24b} are each independently selected from C₁₋₆ alkyl and phenyl, wherein the alkyl is substituted with 0 or 1 phenyl substituents;
optionally R₂₄ₐ and R_{24b} are taken together with the nitrogen to which they are attached to form a piperidine which is substituted with 0, 1 or 2 phenyl substituents;
n is a positive integer from 2 to 4;

and pharmaceutically acceptable salts thereof.

In some embodiments, R₁₇ is hydrogen. In some embodiments, R₁₈ₐ and R_{18b} are each independently C₁₋₆ alkyl, e.g., methyl. In some embodiments, R₁₉ₐ, R_{19b}, R₂₀ₐ, R_{20b}, and R₂₁ are each independently selected from hydrogen and nitro. In some embodiments, R₁₉ₐ, R_{19b}, R₂₀ₐ, and R₂₁ are each independently hydrogen. In some embodiments, R_{20b} is nitro. In some embodiments, R₂₂ is C₁₋₆ alkyl, e.g., methyl.

In some embodiments, the bonds represented by the structure

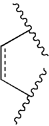

are each independently double bonds.

In some embodiments, R₂₃ is —(CH₂)ₙNR₂₄ₐR_{24b}. In some embodiments, R₂₄ₐ and R_{24b} are each independently C₁₋₆ alkyl substituted with 0 or 1 phenyl substituents. In some embodiments, R₂₄ₐ is C₁₋₆ alkyl, e.g., methyl. In some embodiments, R_{24b} is C₁₋₆ alkyl substituted with 1 phenyl, e.g., benzyl. In some embodiments, R₂₄ₐ and R_{24b} are taken together with the nitrogen to which they are attached to form a piperidine which is substituted with 0, 1 or 2 phenyl substituents. In some embodiments, R₂₄ₐ and R_{24b} are taken together with the nitrogen to which they are attached to form a piperidine which is substituted with 2 phenyl substituents. In some embodiments, n is 2. In some embodiments, n is 3.

In some embodiments, the pharmaceutically acceptable salt is a hydrochloride salt.

In some embodiments, the autophagy inducing compounds of the present invention include the following compounds:

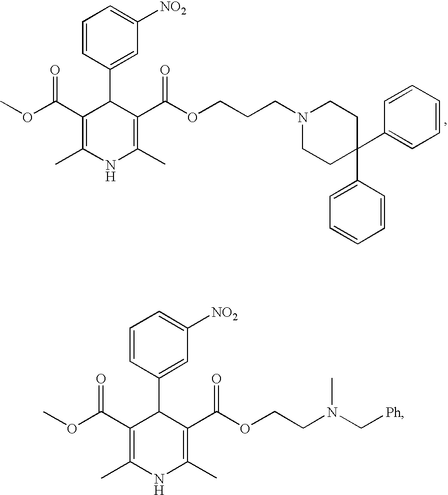

and pharmaceutically acceptable salts thereof.

In some embodiments, the present invention is directed to autophagy inducing compounds of formula (III):

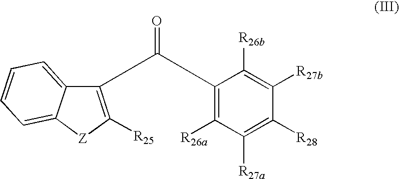

wherein R₂₅ is selected from hydrogen and C₁₋₆ alkyl;
R₂₆ₐ, R_{26b}, R₂₇ₐ, and R_{27b} are each independently selected from hydrogen, halogen and C₁₋₆ alkyl;
R₂₈ is selected from —O(CH₂)ₘNR₂₉ₐR_{29b} and —NH(CH₂)ₘNR₂₉ₐR_{29b};
R₂₉ₐ and R_{29b} are each independently selected from hydrogen and C₁₋₆ alkyl;
Z is O, S or NH;
m is a positive integer from 1 to 3;

and pharmaceutically acceptable salts thereof.

In some embodiments, R₂₅ is C₁₋₆ alkyl, e.g., propyl or butyl. In some embodiments, R₂₆ₐ, R_{26b}, R₂₇ₐ, and R_{27b} are each independently selected from hydrogen and halogen. In some embodiments, R₂₆ₐ and R_{26b} are each independently hydrogen. In some embodiments, R₂₇ₐ and R_{27b} are each independently halogen, e.g., iodine.

In some embodiments, R₂₈ is —O(CH₂)ₘNR₂₉ₐR_{29b}. In some embodiments, R₂₉ₐ and R_{29b} are each independently C₁₋₆ alkyl, e.g., methyl or ethyl. In some embodiments, m is 2. In some embodiments, Z is O.

In some embodiments, the pharmaceutically acceptable salt is a hydrochloride salt.

In some embodiments, the autophagy inducing compounds of the present invention include the following compound:

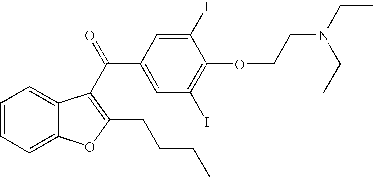

and pharmaceutically acceptable salts thereof.

In some embodiments, the present invention is directed to autophagy inducing compounds of formula (IV):

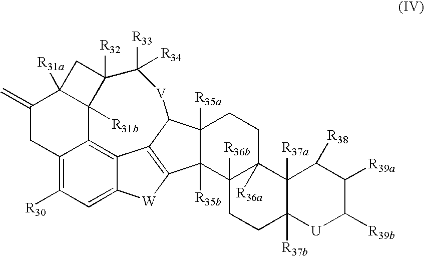

and stereoisomers thereof, e.g., a compound of formula (IV′):

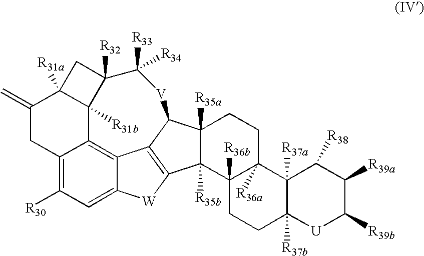

wherein R₃₀ is selected from hydrogen, C₁₋₆ alkyl and halogen;
R₃₁ₐ and R_{31b} are each independently selected from hydrogen, hydroxyl and C₁₋₆ alkyl;
R₃₂ is selected from hydrogen, hydroxyl and C₁₋₆ alkyl;
R₃₃ and R₃₄ are each independently selected from hydrogen and C₁₋₆ alkyl;
R₃₅ₐ and R_{35b} are each independently selected from hydrogen, hydroxyl and C₁₋₆ alkyl;
R₃₆ₐ and R_{36b} are each independently selected from hydrogen, hydroxyl and C₁₋₆ alkyl;
R₃₇ₐ and R_{37b} are each independently selected from hydrogen, hydroxyl and C₁₋₆ alkyl;
R₃₈ is selected from hydrogen, hydroxyl and C₁₋₆ alkyl;
optionally R₃₇ₐ and R₃₈ are taken together to form a three membered heterocycle of the formula:

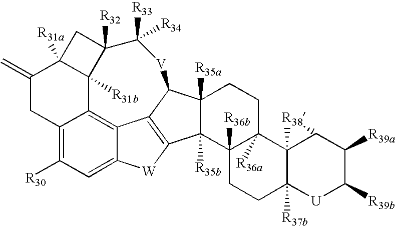

wherein R₃₈′ is O, S or NH;
R₃₉ₐ is selected from hydrogen, hydroxyl and C₁₋₆ alkyl;
R_{39b} is selected from hydrogen, hydroxyl, C₁₋₆ alkyl and C₂₋₆ alkenyl;
U, V and W are each independently selected from 0, S, and NH;

and pharmaceutically acceptable salts thereof.

In some embodiments, R₃₀ is a halogen, e.g., chlorine. In some embodiments, R₃₀ is hydrogen. In some embodiments, R₃₁ₐ and R_{31b} are each independently selected from hydrogen and hydroxyl. In some embodiments, R₃₁ₐ is hydrogen. In some embodiments, R_{31b} is hydroxyl. In some embodiments, R₃₂ is hydrogen. In some embodiments, R₃₃ and R₃₄ are each independently selected from C₁₋₆ alkyl. In some embodiments, R₃₃ is methyl. In some embodiments, R₃₄ is methyl.

In some embodiments, R₃₅ₐ and R_{35b} are each independently selected from hydrogen and C₁₋₆ alkyl. In some embodiments, R₃₅ₐ is hydrogen. In some embodiments, R_{35b} is C₁₋₆ alkyl, e.g., methyl. In some embodiments, R₃₆ₐ and R_{36b} are each independently selected from hydroxyl and C₁₋₆ alkyl. In some embodiments, R₃₆ₐ is hydroxyl. In some embodiments, R_{36b} is C₁₋₆ alkyl, e.g., methyl. In some embodiments, R₃₇ₐ and R_{37b} are each independently selected from hydrogen or hydroxyl. In some embodiments, R₃₈ is selected from hydrogen or hydroxyl. In some embodiments, R₃₇ₐ, R_{37b} and R₃₈ are each independently hydroxyl. In some embodiments, R₃₇ₐ and R₃₈ are taken together to form a three membered heterocycle of the formula:

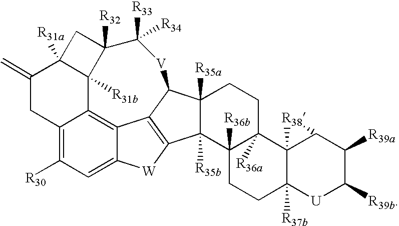

In some embodiments, R₃₈′ is O.

In some embodiments, R₃₉ₐ is hydroxyl. In some embodiments, R_{39b} is selected from C₁₋₆ alkyl and C₂₋₆ alkenyl. In some embodiments, R_{39b} is C₂₋₆ alkenyl, e.g., isobutylene.

In some embodiments, U and V are each independently O, S. In some embodiments, W is NH.

In some embodiments, the pharmaceutically acceptable salt is a hydrochloride salt.

In some embodiments, the autophagy inducing compounds of the present invention include the following compound:

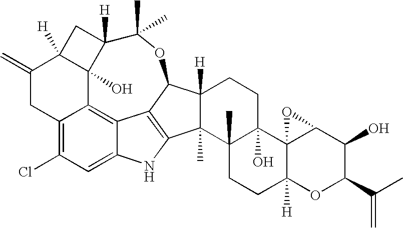

and pharmaceutically acceptable salts thereof.

The autophagy inducing compounds of the present invention promote autophagy and reduce misfolded protein aggregates in the cell. It is interesting that, except for penitrem A, the other compounds described herein exhibit lower cytotoxicity and are superior to rapamycin and to another known autophagy inducing agent, tamoxifen. The foregoing autophagy inducing compounds are useful in the treatment of autophagy associated dieases, such as neurodegenerative diseases caused by mismatched proteins, such as polyglutamine expansion diseases, and can be prepared so as to become better therapeutic drugs for the treatment of diseases caused by misfolded protein aggregates and other autophagy-related diseases.

### F. Therapy

The instant invention features methods for treating autophagy associated diseases, e.g., diseases caused by misfolded protein aggregates, in a subject, by administering to a subject an autophagy inducing compound in an amount effective to treat or prevent the disease.

The methods of the invention further include administering to a subject a therapeutically effective amount of an autophagy inducing compound in combination with another pharmaceutically active compound known to treat an autophagy associated disease; or a compound that may potentiate the autophagy inducing activity of the autophagy inducing compound. Other pharmaceutically active compounds that may be used can be found in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference. The autophagy inducing compound and the additional pharmaceutically active compound(s) may be administered to the subject in the same pharmaceutical composition or in different pharmaceutical compositions (at the same time or at different times).

I. Diseases Caused by Misfolded Protein Aggregates

The methods and compositions of the present invention can be used to treat, for example, Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, and neuronal intranuclear hyaline inclusion disease or any other diseases caused by misfolded protein aggregates described herein.

II. Additional Autophagy-Related Diseases

The methods and compositions of the present invention may also be used to treat other diseases associated with autophagy. Such diseases may include cancer. Preferably, the cancer may be any cancer wherein the induction of autophagy would inhibit cell growth and division, reduce mutagenesis, remove mitochondria and other organelles damaged by reactive oxygen species or kill developing tumor cells. For example, the cancer may be cancer of the breast, liver, prostate, stomach, colon, GI tract, pancreases, skin, head, neck, throat, bladder, eye, esophagus, lung, kidney, or brain.

### G. Pharmaceutical Compositions

The invention features compositions, kits, and methods for treating or preventing a disease or condition associated with diseases caused by misfolded protein aggregates or additional autophagy-related diseases by administering a compound of the invention (i.e., an autophagy inducing compound). Compounds of the present invention may be administered by any appropriate route for treatment or prevention of a disease or condition associated with misfolded protein aggregates or additional autophagy-related diseases. These may be administered to humans, domestic pets, livestock, or other animals with a pharmaceutically acceptable diluent, carrier, or excipient, in unit dosage form. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

Therapeutic formulations may be in the form of liquid solutions or suspensions; for oral administration, formulations may be in the form of tablets or capsules; and for intranasal formulations, in the form of powders, nasal drops, ear drops, or aerosols.

Methods well known in the art for making formulations are found, for example, in “Remington: The Science and Practice of Pharmacy” (20th ed., ed. A. R. Gennaro, 2000, Lippincott Williams & Wilkins). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycholate and deoxycholate, or may be oily solutions for administration in the form of nasal drops, or as a gel. The concentration of the compound in the formulation will vary depending upon a number of factors, including the dosage of the drug to be administered, and the route of administration.

The compound may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

Administration of compounds in controlled release formulations is useful where the autophagy inducing compound has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain the plasma level at a therapeutic level.

Many strategies can be pursued to obtain controlled release in which the rate of release outweighs the rate of metabolism of the therapeutic compound. For example, controlled release can be obtained by the appropriate selection of formulation parameters and ingredients, including, e.g., appropriate controlled release compositions and coatings. Examples include single or multiple unit tablet or capsule compositions, oil solutions, suspensions, emulsions, microcapsules, microspheres, nanoparticles, patches, and liposomes.

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided in unit dosage form as chewable tablets, tablets, caplets, or capsules (i.e., as hard gelatin capsules wherein the active ingredient is mixed with an inert solid diluent, or as soft gelatin capsules wherein the active ingredient is mixed with water or an oil medium).

The formulations can be administered to human subjects in therapeutically effective amounts. Typical dose ranges are from about 0.01 μg/kg to about 2 mg/kg of body weight per day. The preferred dosage of drug to be administered is likely to depend on such variables as the type and extent of the disorder, the overall health status of the particular subject, the specific compound being administered, the excipients used to formulate the compound, and its route of administration. Routine experiments may be used to optimize the dose and dosing frequency for any particular compound.

In one embodiment, the autophagy inducing compound is administered at a concentration in the range from about 0.001 μg/kg to greater than about 500 mg/kg. For example, the concentration may be 0.001 μg/kg, 0.01 μg/kg, 0.05 μg/kg, 0.1 μg/kg, 0.5 μg/kg, 1.0 μg/kg, 10.0 μg/kg, 50.0 μg/kg, 100.0 μg/kg, 500 μg/kg, 1.0 mg/kg, 5.0 mg/kg, 10.0 mg/kg, 15.0 mg/kg, 20.0 mg/kg, 25.0 mg/kg, 30.0 mg/kg, 35.0 mg/kg, 40.0 mg/kg, 45.0 mg/kg, 50.0 mg/kg, 60.0 mg/kg, 70.0 mg/kg, 80.0 mg/kg, 90.0 mg/kg, 100.0 mg/kg, 150.0 mg/kg, 200.0 mg/kg, 250.0 mg/kg, 300.0 mg/kg, 350.0 mg/kg, 400.0 mg/kg, 450.0 mg/kg, to greater than about 500.0 mg/kg or any incremental value thereof. It is to be understood that all values and ranges between these values and ranges are meant to be encompassed by the present invention.

In another embodiment, the autophagy inducing compound is administered in doses that range from 0.01 μM to greater than or equal to 500 μM. For example, the dose may be 0.01 μM, 0.02 μM, 0.05 μM, 0.1 μM, 0.15 μM, 0.2 μM, 0.5 μM, 0.7 μM, 1.0 μM, 3.0 μM, 5.0 μM, 7.0 μM, 10.0 μM, 15.0 μM, 20.0 μM, 25.0 μM, 30.0 μM, 35.0 μM, 40.0 μM, 45.0 μM, 50.0 μM, 60.0 μM, 70.0 μM, 80.0 μM, 90.0 μM, 100.0 μM, 150.0 μM, 200.0 μM, 250.0 μM, 300.0 μM, 350.0 μM, 400.0 μM, 450.0 μM, to greater than about 500.0 μM or any incremental value thereof. It is to be understood that all values and ranges between these values and ranges are meant to be encompassed by the present invention.

In yet another embodiment, the autophagy inducing compound is administered at concentrations that range from 0.10 μg/ml to 500.0 μg/ml. For example, the concentration may be 0.10 μg/ml, 0.50 μg/ml, 1 μg/ml, 2.0 μg/ml, 5.0 μg/ml, 10.0 μg/ml, 20 μg/ml, 25 μg/ml. 30 μg/ml, 35 μg/ml, 40 μg/ml, 45 μg/ml, 50 μg/ml, 60.0 μg/ml, 70.0 μg/ml, 80.0 μg/ml, 90.0 μg/ml, 100.0 μg/ml, 150.0 μg/ml, 200.0 μg/ml, 250.0 g/ml. 250.0 μg/ml, 300.0 μg/ml, 350.0 μg/ml, 400.0 μg/ml, 450.0 μg/ml, to greater than about 500.0 μg/ml or any incremental value thereof. It is to be understood that all values and ranges between these values and ranges are meant to be encompassed by the present invention.

### H. Kits of the Invention

In one aspect, the present invention discloses a kit which includes a pharmaceutical composition comprising an autophagy inducing compound of the present invention and instructions for administering the composition to a subject for the treatment or prevention of an autophagy associated disease, e.g., a disease caused by misfolded protein aggregates. In one embodiment of this aspect, the pharmaceutical composition may include one or more of the following autophagy inducing compounds; Loperamide, Amiodarone, Niguldipine, Pimozide, Nicardipine, Penitrem A, Fluspirilene, or Trifluoerazine. In another embodiment of this aspect, the pharmaceutical composition may comprise a pharmaceutically acceptable carrier.

The present invention is further illustrated by the following examples, which should not be construed as further limiting. The contents of all figures and all references, patents and published patent applications cited throughout this application (as they relate to, for example, the components of the screening methods described herein), as well as the Figures, are expressly incorporated herein by reference in their entirety.

### Examples

### Example 1

### Compounds that Increase LC3-GFP Expression and Accumulation

### Method of Experiment:

Microtubule-associated protein light chain 3 (LC3) is the mammalian protein homologue of yeast autophagy protein ATG8 (Aut7/Apg8) and is positioned on the pre-autophagosome and on the surface of the autophagosome membrane, and was used herein as an autophagosome membrane marker (as described in Mizushima, N. 2004. Int J Biochem Cell Biol. 36: 2491-502). In the instant invention, high-content screening and microscopic analysis was used to assay changes in fluorescent intensity and distribution of GFP labeled LC3 (LC3-GFP) before and after the action of a potential autophagy inducing compound,

### Specific Method:

H4 cells were transfected with the LC3-GFP fusion protein, thereby creating, screening, and yielding an H4-LC3 cell line as a screening platform in order to perform high-content screening of 480 known bioactive compounds (ICCB known bioactive library, BIOMOL), using the following method. DMSO was used to dilute, dissolve, and prepare a compound at different concentrations with each concentration repeated three times. A 96-well plate was inoculated with H4-LC3 cells at an appropriate density and treated for 24 hours with the compound. The following trial groups were established: blank control (treated with DMSO), positive control (treated with inducing agent rapamycin), and a trial group (treated with the compound). If a compound affected LC3 expression or distribution, then this would be embodied precisely in the image and in the size and intensity of fluorescent spots and the like numerical values. This experiment was repeated three times, and the 72 compounds for which LC3-GFP fluorescent intensity was more than 50% greater than that of the blank control group in the three experiments were selected for analysis. Based on the number of cells determined through counting, those compounds in which cell death exceeded 30% (in comparison with the control group) were eliminated. The remaining compounds were further studied as candidate molecules.

### Results of Experiment:

After screening 480 compounds, it was determined that 47 compounds (indicated *) markedly increased LC3-GFP while not causing significant cell death, and could be used in further screening for possible autophagy inhibiting agents. See Table 1 for results:

TABLE 1
Compounds that increased LC3-GFP intensity and are basically not cytotoxic

| | | | | Relative |
|---|---|---|---|---|
| | | | | fluorescent |
| | | Concentration | Relative quantity | intensity of LC3- |
| Compound | Chinese name | (μM) | of cells (%) | GFP (%) |
| Rapamycin | Rapamycin | 0.2 | 88.62 ± 1.47 | 285.38 ± 9.15 |
| Tamoxifen | Tamoxifen | 4.4 | 91.20 ± 15.79 | 585.87 ± 23.60 |
| Grayanotoxin-III* | Grayanotoxin-III | 6.0 | 129.36 ± 12.25 | 210.65 ± 18.76 |
| Loperamide* | Loperamide | 4.9 | 126.76 ± 6.52 | 666.50 ± 29.17 |
| Amiodarone* | Amiodarone | 3.7 | 138.50 ± 1.88 | 327.25 ± 43.47 |
| Bay K-8644* | | 7.0 | 134.45 ± 31.56 | 181.32 ± 24.39 |
| Niguldipine* | Niguldipine | 3.9 | 132.78 ± 18.11 | 777.92 ± 93.57 |
| Pimozide* | Pimozide | 5.4 | 127.14 ± 5.73 | 447.75 ± 27.40 |
| Clozapine* | Clozapine | 7.7 | 139.18 ± 7.07 | 350.13 ± 4.27 |
| Monensin* | Monensin | 3.6 | 92.38 ± 20.28 | 639.29 ± 131.46 |
| Nigericin* | Nigericin | 3.4 | 106.90 ± 20.30 | 747.88 ± 59.06 |
| Wiskostatin* | | 5.9 | 96.96 ± 20.37 | 2401.38 ± 69.13 |
| E6 Berbamine* | Berbamine | 3.3 | 131.16 ± 51.61 | 2447.47 ± 118.04 |
| Paxilline* | | 5.7 | 119.79 ± 14.88 | 205.83 ± 23.64 |
| 2,5- | 2,5- | 11.3 | 118.42 ± 15.99 | 326.89 ± 28.36 |
| Ditertbutylhydroquinone* | Ditertbutylhydroquinone | | | |
| Cyclopiazonic acid* | Cyclopiazonic acid | 7.4 | 112.62 ± 3.20 | 186.44 ± 24.02 |
| Flunarizine* | Flunarizine | 5.2 | 104.67 ± 23.55 | 203.66 ± 5.56 |
| AM 92016* | | 5.2 | 135.28 ± 14.74 | 222.83 ± 4.66 |
| FPL-64176* | | 7.2 | 132.45 ± 18.98 | 200.83 ± 6.35 |
| Verapamil* | Verapamil | 5.2 | 132.44 ± 14.80 | 238.60 ± 7.49 |
| Bepridil* | Bepridil | 6.2 | 111.92 ± 36.16 | 168.39 ± 10.81 |
| Nicardipine* | Nicardipine | 4.8 | 131.58 ± 2.77 | 229.64 ± 11.37 |
| Penitrem A* | Penitrem A | 3.9 | 93.08 ± 14.17 | 166.85 ± 12.90 |
| Propafenone* | Propafenone | 6.6 | 99.88 ± 10.80 | 385.97 ± 11.98 |
| Quinine* | Quinine | 6.9 | 115.16 ± 3.83 | 165.01 ± 12.20 |
| SDZ-201106* | | 5.4 | 94.70 ± 8.45 | 329.96 ± 42.81 |
| Fluspirilene* | Fluspirilene | 5.3 | 143.30 ± 17.53 | 1593.16 ± 23.32 |
| Trifluoperazine* | Trifluoperazine | 8.3 | 105.72 ± 12.84 | 1010.35 ± 109.78 |
| TMB-8* | | 5.8 | 145.22 ± 28.06 | 229.50 ± 8.36 |
| Cyclosporin A* | Cyclosporin A | 2.1 | 159.64 ± 24.07 | 178.79 ± 12.89 |
| Cypermethrin* | Cypermethrin | 6.0 | 97.45 ± 28.70 | 208.14 ± 50.53 |
| NapSul-Ile-Trp- | | 5.1 | 124.74 ± 15.35 | 199.27 ± 27.78 |
| CHO* | | | | |
| CA-074-Me* | | 6.3 | 132.20 ± 1.87 | 272.48 ± 13.64 |
| E-64-d* | | 7.3 | 119.19 ± 21.97 | 244.88 ± 42.95 |
| Ac-Leu-Leu-Nle- | | 6.5 | 96.31 ± 6.90 | 495.29 ± 25.66 |
| CHO* | | | | |
| Calpeptin* | | 6.9 | 128.87 ± 13.59 | 232.89 ± 14.22 |
| Geldanamycin* | Geldanamycin | 4.5 | 81.41 ± 1.67 | 546.80 ± 22.61 |
| Chelerythrine* | Chelerythrine | 6.5 | 110.26 ± 10.99 | 213.88 ± 27.31 |
| BADGE* | | 7.3 | 132.48 ± 3.64 | 182.11 ± 14.16 |
| GW-9662* | | 9.0 | 105.38 ± 6.48 | 166.91 ± 16.53 |
| Castanospermine* | Castanospermine | 13.2 | 157.35 ± 11.87 | 171.20 ± 10.92 |
| Dipyridamole* | Dipyridamole | 5.0 | 158.96 ± 20.49 | 213.63 ± 8.98 |
| CAPE* | | 8.8 | 146.20 ± 21.95 | 150.18 ± 24.10 |
| GM6001* | | 6.4 | 143.94 ± 11.51 | 196.42 ± 24.22 |
| H9* | | 7.71 | 96.70 ± 4.54 | 156.16 ± 13.15 |
| K252A* | | 0.5 | 101.89 ± 3.64 | 215.29 ± 17.56 |
| Indirubin* | Indirubin | 9.5 | 101.26 ± 13.87 | 172.28 ± 37.92 |
| 24(S)- | 24(S)- | 6.2 | 108.20 ± 15.82 | 648.90 ± 135.44 |
| Hydroxycholesterol* | Hydroxycholesterol | | | |
| Cyclopamine* | Cyclopamine | 6.1 | 110.76 ± 20.93 | 1564.12 ± 149.37 |
| SB 202190 | | 7.6 | 91.14 ± 27.00 | 169.71 ± 31.55 |
| ML9 | | 6.9 | 82.33 ± 14.75 | 447.81 ± 36.14 |
| Cytochalasin D | Cytochalasin D | 4.9 | 71.84 ± 23.37 | 378.77 ± 59.48 |
| Bafilomycin A1 | Bafilomycin A1 | 0.4 | 71.96 ± 7.74 | 4027.54 ± 133.76 |
| Tanshinone IIA | Tanshinone IIA | 8.5 | 79.77 ± 21.67 | 273.72 ± 21.09 |
| Aphidicolin | Aphidicolin | 7.4 | 73.06 ± 11.61 | 350.13 ± 4.27 |
| 17-Allylamino- | 17-Allylamino- | 4.3 | 66.89 ± 1.23 | 385.49 ± 35.56 |
| geldanamycin | geldanamycin | | | |
| Ikarugamycin | Ikarugamycin | 5.2 | 50.16 ± 5.27 | 172.85 ± 35.93 |
| Latrunculin B | | 6.3 | 27.55 ± 3.99 | 723.64 ± 78.60 |
| Trichostatin-A | Trichostatin-A | 5.2 | 40.15 ± 5.26 | 1649.69 ± 174.70 |
| Thapsigargin | Thapsigargin | 3.8 | 63.77 ± 8.62 | 187.15 ± 2.47 |
| A-23187 | | 4.8 | 49.30 ± 3.18 | 150.83 ± 14.52 |
| SKF-96365 | | 6.2 | 42.96 ± 4.32 | 285.59 ± 17.28 |
| Ro 31-8220 | | 4.5 | 50.58 ± 12.18 | 240.67 ± 33.17 |
| GF-109203X | | 6.1 | 47.77 ± 2.96 | 211.81 ± 18.29 |
| Cytochalasin B | Cytochalasin B | 5.2 | 30.58 ± 18.88 | 286.43 ± 20.43 |
| Cantharidin | Cantharidin | 12.7 | 24.47 ± 2.33 | 463.81 ± 22.89 |
| Etoposide | Etoposide | 4.3 | 48.17 ± 4.58 | 335.90 ± 53.85 |
| ICRF-193 | | 8.9 | 45.03 ± 5.04 | 483.18 ± 27.44 |
| Furoxan | | 13.4 | 33.47 ± 4.82 | 214.71 ± 51.52 |
| Curcumin | Curcumin | 6.8 | 59.32 ± 23.06 | 249.22 ± 7.06 |
| OBAA | | 5.8 | 35.84 ± 23.94 | 155.80 ± 23.68 |
| Z-Leu3-VS | | 4.5 | 38.56 ± 5.36 | 176.02 ± 12.29 |

### Example 2

### Compounds that Increase or do not Affect Intracellular PI(3)P Levels

### Method of Experiment:

Class III PI(3)K Vps34 is a multifunctional protein. On the one hand, it catalyzes the phosphorylation of PI to generate PI(3)P and is of key importance in the endocytic and autophagosome membrane transport processes. At the same time, a compound formed from Vps34/beclin1 is involved in the regulation of autophagy initiation signaling. Therefore, while the autophagy process is taking place, the level of PtdIns(3)P should not be expected to decrease to a significant extent. A FYVE domain is a protein structure domain composed of approximately 70 amino acid residues and containing a zinc finger protein structure; it can bind specifically with PI(3)P. Generally, PI(3)P recruits proteins containing a FYVE domain in order to bind to the membrane of cellular organelles, and participate in protein trafficking and similar processes.

H4 cells were transfected with the fusion protein FYVP-red fluorescent protein (FYVE-RFP). After transfection, the cells were screened in order to obtain a suitable H4-FYVE cell line to be used as a screening platform to perform high-volume screening of compounds, which thereby indirectly reflected the effect of the compound on autophagy. Compounds which reduced FYVE-RFP fluorescence intensity were eliminated. In this experiment, change in the intensity, fluorescence and distribution of the protein marker FYVE-RFP recruited by PI(3)P before and after treatment with the potential autophagy inducing compound was assayed by high-content microscopic analysis.

### Specific Method:

DMSO was used to dilute, dissolve, and prepare a compound at different concentrations with each concentration repeated three times. A 96-well plate was inoculated with H4-FYVE cells at an appropriate density and treated with the compound for 2, 4, and 8 hours, respectively. The following trial groups were established: blank control (treated with DMSO), positive control (treated with inducing agent rapamycin), negative control (treated with PI(3)K inhibiting agent LY-294002), and a trial group (treated with the compound). If a compound affected FYVE expression or distribution, then this would be embodied precisely in the image and in the size and intensity of fluorescent spots and other numerical values. This experiment was repeated three times, and the compounds that markedly reduced FYVE were eliminated. The various compounds for which RFP-FYVE fluorescent intensity showed no marked reduction in comparison with the blank control group in the three experiments were selected for further analysis.

### Results of Experiment:

After screening 47 compounds, it was determined that 26 compounds did not significantly reduce FYVE-RFP (including the eight compounds given above), and could be used in further screening for possible autophagy inhibiting agents. See Table 2:

TABLE 2
Compounds that did not decrease FYVE-
RFP expression or accumulation

| | Relative fluorescent intensity of FYVE-RFP (%) | | |
|---|---|---|---|
| Compound | 2 h | 4 h | 8 h |
| Rapamycin | 164.93 ± 10.96 | 131.41 ± 26.64 | 152.35 ± 4.55 |
| Nigericin | 148.43 ± 5.17 | 145.40 ± 8.39 | 140.54 ± 18.21 |
| Wiskostatin | 267.90 ± 6.16 | 240.08 ± 4.26 | 131.93 ± 11.05 |
| Fluspirilene | 224.98 ± 16.90 | 142.15 ± 5.77 | 108.53 ± 6.07 |
| Niguldipine | 147.18 ± 25.39 | 148.26 ± 3.67 | 125.53 ± 2.84 |
| Trifluoperazine | 136.58 ± 15.74 | 111.52 ± 13.45 | 94.28 ± 2.87 |
| Nicardipine | 132.05 ± 19.93 | 112.51 ± 10.09 | 104.77 ± 31.63 |
| Penitrem A | 121.40 ± 12.66 | 87.41 ± 5.90 | 72.71 ± 5.57 |
| Tamoxifen | 77.66 ± 4.43 | 71.55 ± 0.73 | 85.37 ± 7.59 |
| Loperamide | 115.39 ± 5.20 | 123.62 ± 1.37 | 95.70 ± 12.84 |
| Amiodarone | 92.50 ± 5.51 | 89.23 ± 3.47 | 83.45 ± 3.64 |
| Pimozide | 94.42 ± 4.71 | 103.96 ± 9.06 | 73.64 ± 12.18 |
| Clozapine | 64.04 ± 2.94 | 74.79 ± 13.43 | 78.38 ± 1.35 |
| Cyclopamine | 108.75 ± 12.44 | 75.21 ± 1.18 | 96.38 ± 5.12 |
| Paxilline | 100.56 ± 0.58 | 52.37 ± 1.65 | 56.82 ± 4.44 |
| FPL-64176 | 99.43 ± 9.62 | 78.17 ± 1.90 | 87.62 ± 2.42 |
| Verapamil | 83.96 ± 20.67 | 70.15 ± 13.00 | 71.28 ± 11.29 |
| Propafenone | 114.69 ± 8.90 | 78.75 ± 18.93 | 62.87 ± 3.38 |
| Bay K-8644 | 110.18 ± 25.18 | 57.79 ± 3.76 | 79.70 ± 7.28 |
| Quinine | 70.35 ± 10.23 | 78.03 ± 6.33 | 60.70 ± 6.59 |
| SDZ-201106 | 65.87 ± 4.72 | 59.79 ± 2.38 | 72.40 ± 20.27 |
| TMB-8 | 89.59 ± 4.79 | 74.72 ± 13.74 | 78.64 ± 10.26 |
| Cyclosporin A | 73.62 ± 5.70 | 71.69 ± 27.87 | 55.82 ± 4.16 |
| NapSul-Ile-Trp-CHO | 89.37 ± 3.52 | 64.10 ± 11.47 | 62.34 ± 8.21 |
| CA-074-Me | 65.94 ± 12.73 | 79.82 ± 4.66 | 86.43 ± 4.15 |
| Ac-Leu-Leu-Nle-CHO | 72.86 ± 10.18 | 84.85 ± 2.83 | 88.42 ± 6.29 |
| CAPE | 86.89 ± 20.98 | 71.17 ± 18.70 | 69.14 ± 4.32 |
| H9 | 102.35 ± 12.94 | 79.63 ± 1.52 | 83.01 ± 6.62 |
| K252A | 76.86 ± 6.51 | 65.58 ± 9.36 | 70.33 ± 5.89 |
| AM 92016 | 68.06 ± 1.20 | 71.97 ± 15.67 | 66.35 ± 17.66 |

### Example 3

### Compounds that Increase the Degradation of Long-Lived Proteins

### Method of Experiment:

The process of autophagy is a protein degradation process, which primarily mediates the degradation of cellular organelles and long-lived proteins within the cell. Therefore, in the present experiment the detection of whether a compound promotes the degradation of long-lived proteins within the cell was used as an additional indicator of whether the tested compound induces autophagy.

### Specific Method:

DMSO was used to dilute, dissolve, and prepare a compound at different concentrations with each concentration repeated three times. A 96-well plate was inoculated with 114 cells at an appropriate density and treated with the compound for 2, 4, and 24 hours, respectively. The following trial groups were established: blank control (treated with DMSO), positive control (treated with inducing agent rapamycin), and a trial group (treated with the compound). One day prior to the experiment, cells were inoculated onto a 12-well plate at an appropriate density and cultured under normal conditions (DMEM+10% fetal calf serum). Prior to the experiment, the culture medium was replaced with a complete medium that did not contain L-leucine and cultured for 1 hour in order to eliminate endogenous L-leucine within the cells. Then, a complete medium containing (3H) L-leucine was used to incubate the cells for 24 hours causing the cells to take in isotope-labeled leucine and undertake protein synthesis. After 24 hours, a complete culture medium was substituted and used to incubate the cells for 24 hours to degrade short-lived proteins. After the short-lived proteins had been degraded, a complete culture medium to which the compound had been added was substituted and used to incubate the cells. The radiation intensity of the culture medium was tested at 0, 1, 2, 4, and 24 hours, respectively. At 24 hours, the cells were collected and the intensity of the isotope within the cells was tested, and the percent of long-lived protein degradation relative to the blank control group at different times after the action of the compound was computed. Compounds that increased the degradation of long-lived proteins were selected by means of t-test.

### Results of Experiment:

Eight compounds that increase the degradation of long-lived proteins (promote autophagy) were found. See Table 3:

TABLE 3
Compounds that promote the degradation of long-lived proteins

| | Percent relative degradation of long-lived proteins (%) | | | |
|---|---|---|---|---|
| Compound | 1 h | 2 h | 4 h | 24 h |
| Rapamycin | 159.89 ± 11.46 | 171.67 ± 10.41 | 149.89 ± 24.83 | 165.87 ± 4.08 |
| Loperamide | 78.70 ± 13.17 | 122.10 ± 6.48 | 125.21 ± 4.29 | 139.19 ± 18.77 |
| Fluspirilene | 93.82 ± 3.25 | 143.17 ± 4.26 | 144.79 ± 9.02 | 145.50 ± 2.98 |
| Trifluoperazine | 76.62 ± 2.32 | 105.60 ± 5.01 | 109.00 ± 5.22 | 124.78 ± 2.05 |
| Pimozide | 129.13 ± 11.46 | 155.80 ± 9.22 | 152.01 ± 9.63 | 162.47 ± 3.50 |
| Nicardipine | 84.62 ± 4.48 | 126.59 ± 3.83 | 122.60 ± 7.70 | 121.03 ± 13.43 |
| Penitrem A | 92.88 ± 2.83 | 126.09 ± 0.47 | 132.13 ± 10.01 | 141.83 ± 1.25 |
| Niguldipine | 71.65 ± 2.68 | 107.42 ± 2.72 | 105.68 ± 2.74 | 117.85 ± 1.98 |
| Amiodarone | 101.32 ± 5.95 | 122.42 ± 9.71 | 110.75 ± 3.68 | 116.73 ± 5.54 |

### Example 4

### Compounds that Increase Cell's Autophagy-Related Markings and LC3 II Ratios

### Method of Experiment:

The autophagosome marker LC3 used in the compound-screening platform was subjected to the foregoing processing in the cytosol. Generally when the new LC3 synthesized within the cell was processed, it became cytosolic soluble LC3 I with molecular weight 18 Kd. When autophagy occurs, after the latter undergoes modification through ubiquitin-like processing and binds with phosphatidylethanolamine (PE) on the autophagosomal membrane surface, it is called LC3 II, which is localized on the autophagosomal membrane and has apparent molecular weight 16 KD (Kabeya, Y., Mizushima, N., Ueno, T., et al. 2000. EMBO J. 19: 5720-8). The quantity of LC3 II content reflects to a certain degree the autophagic activity of a cell. Therefore, by assaying the content of LC3 II in the cell by means of the Western blot method, it is possible to further reflect the effect of the compound on autophagy.

### Specific Method:

H4-LC cells at an appropriate density were inoculated onto a 6-well plate, which was treated with a compound for 4 hours, after which the cells were collected and lysed. The protein fraction was harvested and SDS-PAGE was performed. Following electrophoresis separation the proteins were transferred to a membrane, and immunoblot analysis was performed. The cytoskeleton protein, actin, was used as an internal control. The following trial groups were established: negative control (treated with DMSO), positive control (treated with inducing agent rapamycin), and a trial group (treated with the compound).

### Results of the Experiment:

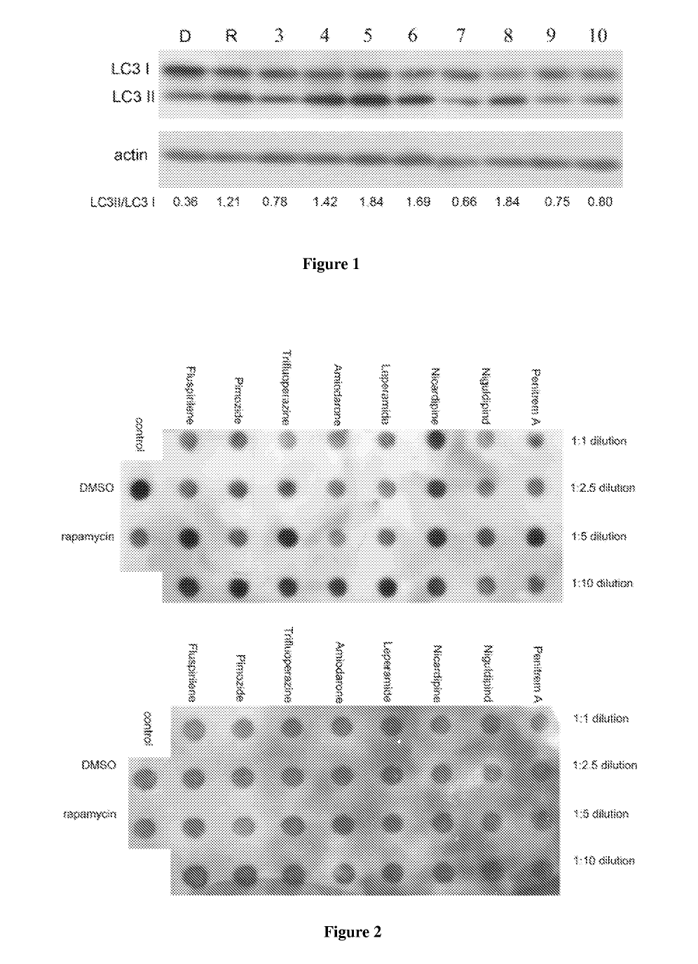

Eight compounds that induce an increase in LC3 II/LC3 I percentage were identified, as shown in FIG. 1.

### Example 5

### Compounds that Promote Degradation of Expanded Polyglutamine

### Method of Experiment:

Large-scale accumulation of misfolded protein is a prominent feature of many neurodegenerative diseases. For example, the pathogenic mechanism of Huntington's disease is that a large amount of polyglutamine (polyQ) protein accumulates in the neurons and cannot be cleared away. Tests of polyQ elimination and degradation were used in this experiment to further determine the ability if the tested compounds to induce autophagy.

### Specific Method:

The compounds selected through screening were dissolved in DMSO and diluted in a stepwise manner in complete culture medium. 114 cells were inoculated onto a 12-well plate at an appropriate density, the cells were transfected with liposome-encapsulated recombinant plasmid GFP-polyQ-HA; after four hours, the medium was replaced using the above-mentioned complete culture medium to which different concentrations of the compound had been added and culturing was continued; after 24 hours, the cells were photographed and collected; after lysis, the protein in the cytosol was harvested and spotted onto a PVDF membrane using a spot applicator, western blot staining was performed using anti-HA antibodies, and the cytoskeleton protein actin was used as an internal control. The following trial groups were established: negative control (treated with DMSO), positive control (treated with inducing agent rapamycin), and a trial group (treated with the compound).

### Results of the Experiment:

As shown in FIG. 2, the eight compounds effectively induce polyQ degradation and exhibit a relatively good dose-dependent relationship and can be prepared into better drugs to treat diseases caused by misfolded protein aggregates and other autophagy-related diseases.

1. A pharmaceutical composition comprising an autophagy inducing compound in an amount effective for treating an autophagy associated disease, wherein said compound is selected from the group consisting of: 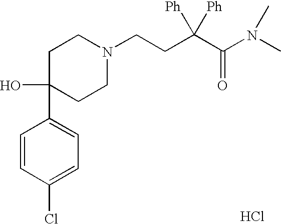
(a) Loperamide; 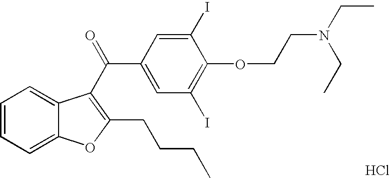
(b) Amiodarone; 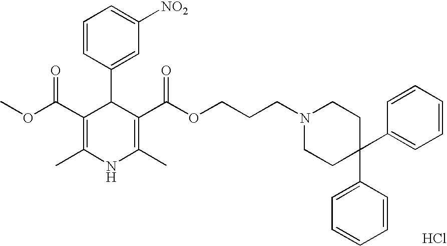
(c) Niguldipine; 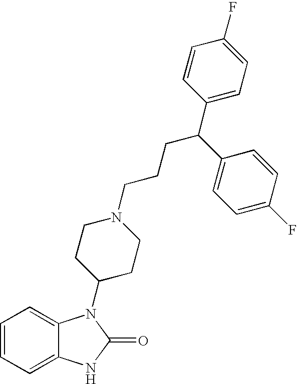
(d) Pimozide; 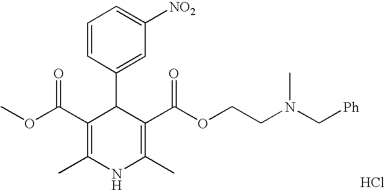
(e) Nicardipine; 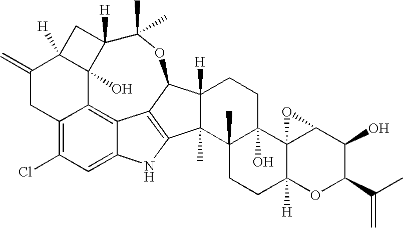
(f) Penitrem A; 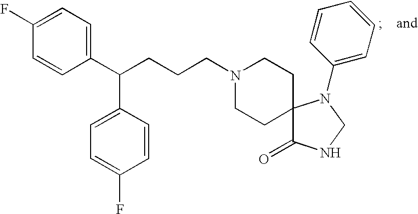
(g) Fluspirilene 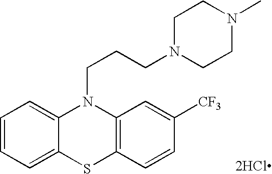
(h) Trifluoperazine.

2. The pharmaceutical composition of claim 1, wherein said composition further comprises a pharmaceutically acceptable carrier.

3. The pharmaceutical composition of claim 1, wherein said autophagy associated disease is a disease caused by misfolded protein aggregates.

4. A pharmaceutical composition comprising an autophagy inducing compound in an amount effective for treating an autophagy associated disease, wherein the compound is at least one compound selected from the group consisting of:
(a) compounds of formula (I): 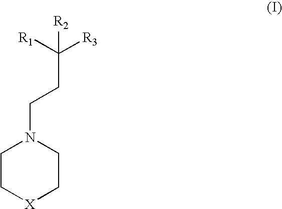
wherein X is selected from CR₄R₅ and NR₆;
R₁ is selected from hydrogen, C₁₋₆ alkyl and phenyl, wherein the alkyl and phenyl are substituted with 0 or 1 halogen;
R₂ is selected from hydrogen, C₁₋₆ alkyl and phenyl, wherein the alkyl and phenyl are substituted with 0 or 1 halogen; 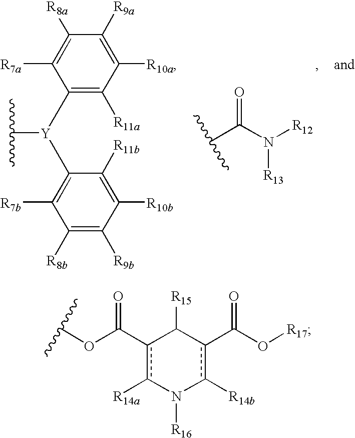
R₃ is selected from
R₄ is selected from hydrogen, hydroxyl, C₁₋₆ alkyl and phenyl;
R₅ is selected from C₁₋₆ alkyl and phenyl, halophenyl, benzimidazole, dihydrobenzimidazole, benzimidazolone;
optionally R₄ and R₅ are taken together to form a 5 or 6 membered heterocycloalkyl comprising two nitrogen atoms, wherein the heterocycloalkyl is substituted with 1, 2 or 3 substituents selected from the group consisting of C₁₋₆ alkyl, phenyl, and ═O;
R₆ is selected from hydrogen and C₁₋₆ alkyl;
R₇ₐ, R₈ₐ, R₉ₐ, R₁₀ₐ, R₁₁ₐ, R_{7b}, R_{8b}, R_{9b}, R_{10b}, and R_{11b} are each independently selected from hydrogen, hydroxyl, halogen and C₁₋₆ haloalkyl; optionally R₁₁ₐ and R_{11b} are taken together to form a heterocycle of the following structure: 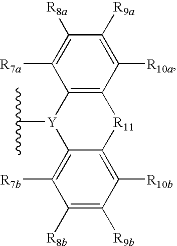
wherein R₁₁ is selected from CH₂, NH, O and S;
R₁₂ and R₁₃ are each independently selected from hydrogen and C₁₋₆ alkyl;
R₁₄ₐ and R_{14b} are each independently selected from hydrogen and C₁₋₆ alkyl;
R₁₅ is selected from phenyl substituted with 0 or 1 halogen or nitro;
R₁₆ is selected from hydrogen and C₁₋₆ alkyl;
Y is N or CH;
and pharmaceutically acceptable salts thereof;
(b) compounds of formula (II): 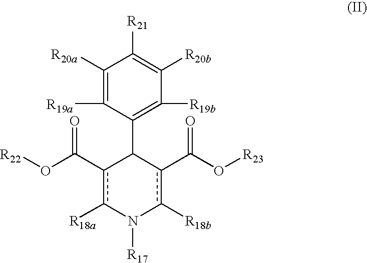
wherein R₁₇ is selected from hydrogen and C₁₋₆ alkyl;
R₁₈ₐ and R_{18b}, are each independently selected from hydrogen and C₁₋₆ alkyl;
R₁₉ₐ, R_{19b}, R₂₀ₐ, R_{20b}, and R₂₁ are each independently selected from hydrogen, halogen and nitro;
R₂₂ is selected from hydrogen and C₁₋₆ alkyl;
R₂₃ is selected from —(CH₂)ₙNR₂₄ₐR_{24b} and —(CH₂)ₙR₂₄ₐ;
R₂₄ₐ and R_{24b} are each independently selected from C₁₋₆ alkyl and phenyl, wherein the alkyl is substituted with 0 or 1 phenyl substituents;
optionally R₂₄ₐ and R_{24b} are taken together with the nitrogen to which they are attached to form a piperidine which is substituted with 0, 1 or 2 phenyl substituents;
n is a positive integer from 2 to 4;
and pharmaceutically acceptable salts thereof
(c) compounds of formula (III): 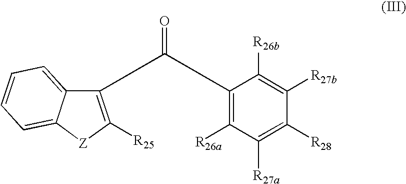
wherein R₂₅ is selected from hydrogen and C₁₋₆ alkyl;
R₂₆ₐ, R_{26b}, R₂₇ₐ, and R_{27b} are each independently selected from hydrogen, halogen and C₁₋₆ alkyl;
R₂₈ is selected from —O(CH₂)ₘNR₂₉ₐR_{29b} and —NH(CH₂)ₘNR₂₉ₐR_{29b};
R₂₉ₐ and R_{29b} are each independently selected from hydrogen and C₁₋₆ alkyl;
Z is O, S or NH;
m is a positive integer from 1 to 3;
and pharmaceutically acceptable salts thereof
(d) compounds of formula (IV): 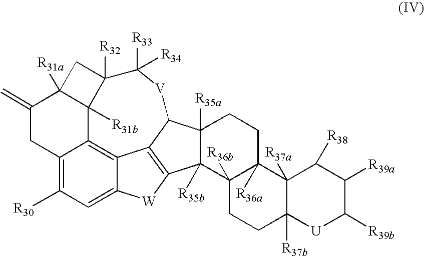
wherein R₃₀ is selected from hydrogen, C₁₋₆ alkyl and halogen;
R₃₁ₐ and R_{31b} are each independently selected from hydrogen, hydroxyl and C₁₋₆ alkyl;
R₃₂ is selected from hydrogen, hydroxyl and C₁₋₆ alkyl;
R₃₃ and R₃₄ are each independently selected from hydrogen and C₁₋₆ alkyl;
R₃₅ₐ and R_{35b} are each independently selected from hydrogen, hydroxyl and C₁₋₆ alkyl;
R₃₆ₐ and R_{36b} are each independently selected from hydrogen, hydroxyl and C₁₋₆ alkyl;
R₃₇ₐ and R_{37b} are each independently selected from hydrogen, hydroxyl and C₁₋₆ alkyl;
R₃₈ is selected from hydrogen, hydroxyl and C₁₋₆ alkyl;
optionally R₃₇ₐ and R₃₈ are taken together to form a three membered heterocycle of the formula: 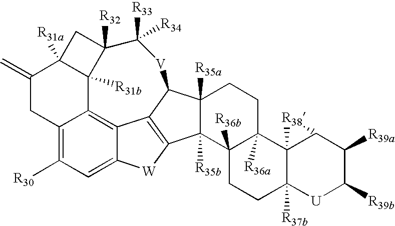
wherein R₃₈′ is O, S or NH;
R₃₉ₐ is selected from hydrogen, hydroxyl and C₁₋₆ alkyl;
R_{39b} is selected from hydrogen, hydroxyl, C₁₋₆ alkyl and C₂₋₆ alkenyl;
U, V and W are each independently selected from O, S, and NH;
and pharmaceutically acceptable salts thereof.

5. The pharmaceutical composition of claim 4, wherein said composition further comprises a pharmaceutically acceptable carrier.

6. The pharmaceutical composition of claim 4, wherein said autophagy associated disease is a disease caused by misfolded protein aggregates.

7. A method of inducing autophagy in a cell, said method comprising contacting said cell with an autophagy inducing compound in an amount effective to induce autophagy in said cell.

8. The method of claim 7, wherein said autophagy inducing compound is selected from the group consisting of Loperamide, Amiodarone, Niguldipine, Pimozide, Nicardipine, Penitrem A, Fluspirilene, and Trifluoperazine and pharmaceutically acceptable salts thereof.

9. The method of claim 7, wherein the compound is at least one compound selected from the group consisting of:
(a) compounds of formula (I): 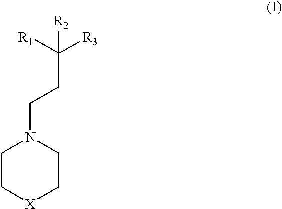
wherein X is selected from CR₄R₅ and NR₆;
R₁ is selected from hydrogen, C₁₋₆ alkyl and phenyl, wherein the alkyl and phenyl are substituted with 0 or 1 halogen;
R₂ is selected from hydrogen, C₁₋₆ alkyl and phenyl, wherein the alkyl and phenyl are substituted with 0 or 1 halogen; 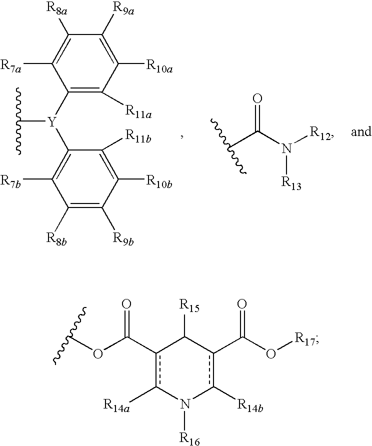
R₃ is selected from
R₄ is selected from hydrogen, hydroxyl, C₁₋₆ alkyl and phenyl;
R₅ is selected from C₁₋₆ alkyl and phenyl, halophenyl, benzimidazole, dihydrobenzimidazole, benzimidazolone;
optionally R₄ and R₅ are taken together to form a 5 or 6 membered heterocycloalkyl comprising two nitrogen atoms, wherein the heterocycloalkyl is substituted with 1, 2 or 3 substituents selected from the group consisting of C₁₋₆ alkyl, phenyl, and ═O;
R₆ is selected from hydrogen and C₁₋₆ alkyl;
R₇ₐ, R₈ₐ, R₉ₐ, R₁₀ₐ, R₁₁ₐ, R_{7b}, R_{8b}, R_{9b}, R_{10b}, and R_{11b} are each independently selected from hydrogen, hydroxyl, halogen and C₁₋₆ haloalkyl; optionally R₁₁ₐ and R_{11b} are taken together to form a heterocycle of the following structure: 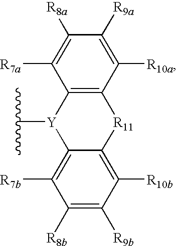
wherein R₁₁ is selected from CH₂, NH, O and S;
R₁₂ and R₁₃ are each independently selected from hydrogen and C₁₋₆ alkyl;
R₁₄ₐ and R_{14b} are each independently selected from hydrogen and C₁₋₆ alkyl;
R₁₅ is selected from phenyl substituted with 0 or 1 halogen or nitro;
R₁₆ is selected from hydrogen and C₁₋₆ alkyl;
Y is N or CH;
and pharmaceutically acceptable salts thereof;
(b) compounds of formula (II): 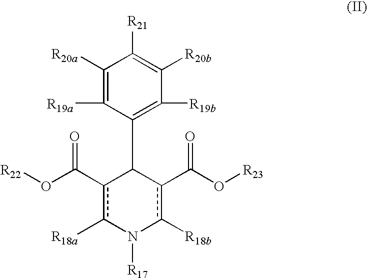
wherein R₁₇ is selected from hydrogen and C₁₋₆ alkyl;
R₁₈ₐ and R_{18b}, are each independently selected from hydrogen and C₁₋₆ alkyl;
R₁₉ₐ, R_{19b}, R₂₀ₐ, R_{20b}, and R₂₁ are each independently selected from hydrogen, halogen and nitro;
R₂₂ is selected from hydrogen and C₁₋₆ alkyl;
R₂₃ is selected from —(CH₂)ₙNR₂₄ₐR_{24b} and —(CH₂)ₙR₂₄ₐ;
R₂₄ₐ and R_{24b} are each independently selected from C₁₋₆ alkyl and phenyl, wherein the alkyl is substituted with 0 or 1 phenyl substituents;
optionally R₂₄ₐ and R_{24b} are taken together with the nitrogen to which they are attached to form a piperidine which is substituted with 0, 1 or 2 phenyl substituents;
n is a positive integer from 2 to 4;
and pharmaceutically acceptable salts thereof
(c) compounds of formula (III): 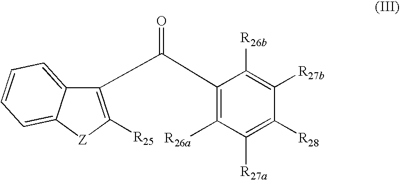
wherein R₂₅ is selected from hydrogen and C₁₋₆ alkyl;
R₂₆ₐ, R_{26b}, R₂₇ₐ, and R_{27b} are each independently selected from hydrogen, halogen and C₁₋₆ alkyl;
R₂₈ is selected from —O(CH₂)ₘNR₂₉ₐR_{29b} and —NH(CH₂)ₘNR₂₉ₐR_{29b};
R₂₉ₐ and R_{29b} are each independently selected from hydrogen and C₁₋₆ alkyl;
Z is O, S or NH;
m is a positive integer from 1 to 3;
and pharmaceutically acceptable salts thereof
(d) compounds of formula (IV): 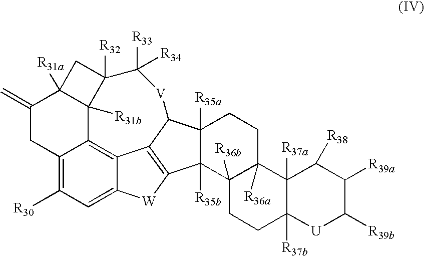
wherein R₃₀ is selected from hydrogen, C₁₋₆ alkyl and halogen;
R₃₁ₐ and R_{31b} are each independently selected from hydrogen, hydroxyl and C₁₋₆ alkyl;
R₃₂ is selected from hydrogen, hydroxyl and C₁₋₆ alkyl;
R₃₃ and R₃₄ are each independently selected from hydrogen and C₁₋₆ alkyl;
R₃₅ₐ and R_{35b} are each independently selected from hydrogen, hydroxyl and C₁₋₆ alkyl;
R₃₆ₐ and R_{36b} are each independently selected from hydrogen, hydroxyl and C₁₋₆ alkyl;
R₃₇ₐ and R_{37b} are each independently selected from hydrogen, hydroxyl and C₁₋₆ alkyl;
R₃₈ is selected from hydrogen, hydroxyl and C₁₋₆ alkyl;
optionally R₃₇ₐ and R₃₈ are taken together to form a three membered heterocycle of the formula: 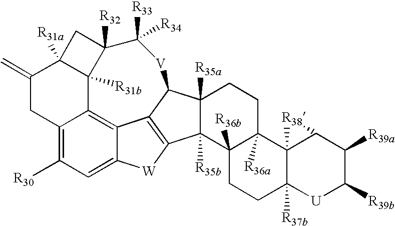
wherein R₃₈′ is O, S or NH;
R₃₉ₐ is selected from hydrogen, hydroxyl and C₁₋₆ alkyl;
R_{39b} is selected from hydrogen, hydroxyl, C₁₋₆ alkyl and C₂₋₆ alkenyl;
U, V and W are each independently selected from O, S, and NH;
and pharmaceutically acceptable salts thereof.

10. The method of claim 7, wherein said cell is present in a subject.

11. The method of claim 7, wherein said cell is present in an in vitro cell culture.

12. (canceled)

13. (canceled)

14. The method of claim 7, wherein said cell is selected from the group consisting of neural cells, glial cells, such as astrocytes, oligodendrocytes, ependymal cells, Schwann cells, lymphatic cells, epithelial cells, endothelial cells, lymphocytes, cancer cells, and haematopoietic cells.

15. A method of treating an autophagy associated disease in a subject, said method comprising administering to said subject an autophagy inducing compound in an amount effective to treat said disease, thereby treating said disease in said subject.

16. The method of claim 15, wherein said autophagy associate disease is a disease caused by misfolded protein aggregates.

17. The method of claim 16, wherein said disease caused by misfolded protein aggregates is selected from the group consisting of Alzheimer's disease, Parkinson's disease, amyotrophic lateral sclerosis, Huntington's disease, spinocerebellar ataxia, oculopharyngeal muscular dystrophy, prion diseases, fatal familial insomnia, alpha-1 antitrypsin deficiency, dentatorubral pallidoluysian atrophy, frontal temporal dementia, progressive supranuclear palsy, x-linked spinobulbar muscular atrophy, and neuronal intranuclear hyaline inclusion disease.

18. The method of claim 16, wherein said disease associated with misfolded protein aggregates is a chronic disease.

19. The method of claim 15, wherein said autophagy associated disease is cancer.

20. The method of claim 15, wherein said autophagy inducing compound is selected from the group comprising Loperamide, Amiodarone, Niguldipine, Pimozide, Nicardipine, Penitrem A, Fluspirilene, Trifluoperazine, and pharmaceutically acceptable salts thereof.

21. The method of claim 15, wherein the compound is at least one compound selected from the group consisting of:
(a) compounds of formula (I): 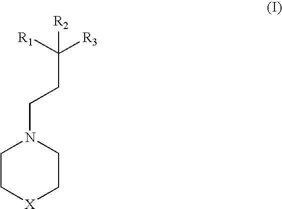
wherein X is selected from CR₄R₅ and NR₆;
R₁ is selected from hydrogen, C₁₋₆ alkyl and phenyl, wherein the alkyl and phenyl are substituted with 0 or 1 halogen;
R₂ is selected from hydrogen, C₁₋₆ alkyl and phenyl, wherein the alkyl and phenyl are substituted with 0 or 1 halogen;
R₃ is selected from 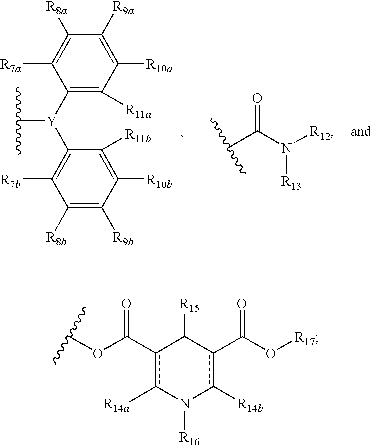
R₄ is selected from hydrogen, hydroxyl, C₁₋₆ alkyl and phenyl;
R₅ is selected from C₁₋₆ alkyl and phenyl, halophenyl, benzimidazole, dihydrobenzimidazole, benzimidazolone;
optionally R₄ and R₅ are taken together to form a 5 or 6 membered heterocycloalkyl comprising two nitrogen atoms, wherein the heterocycloalkyl is substituted with 1, 2 or 3 substituents selected from the group consisting of C₁₋₆ alkyl, phenyl, and ═O;
R₆ is selected from hydrogen and C₁₋₆ alkyl;
R₇ₐ, R₈ₐ, R₉ₐ, R₁₀ₐ, R₁₁ₐ, R_{7b}, R_{8b}, R_{9b}, R_{10b}, and R_{11b} are each independently selected from hydrogen, hydroxyl, halogen and C₁₋₆ haloalkyl; optionally R₁₁ₐ and R_{11b} are taken together to form a heterocycle of the following structure: 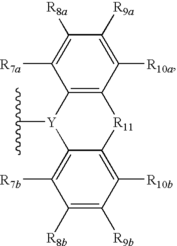
wherein R₁₁ is selected from CH₂, NH, O and S;
R₁₂ and R₁₃ are each independently selected from hydrogen and C₁₋₆ alkyl;
R₁₄ₐ and R_{14b} are each independently selected from hydrogen and C₁₋₆ alkyl;
R₁₅ is selected from phenyl substituted with 0 or 1 halogen or nitro;
R₁₆ is selected from hydrogen and C₁₋₆ alkyl;
Y is N or CH;
and pharmaceutically acceptable salts thereof;
(b) compounds of formula (II): 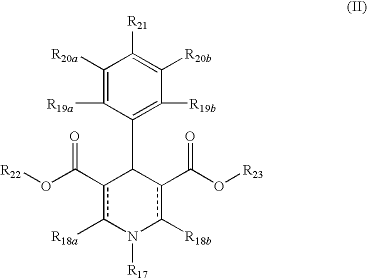
wherein R₁₇ is selected from hydrogen and C₁₋₆ alkyl;
R₁₈ₐ and R_{18b}, are each independently selected from hydrogen and C₁₋₆ alkyl;
R₁₉ₐ, R_{19b}, R₂₀ₐ, R_{20b}, and R₂₁ are each independently selected from hydrogen, halogen and nitro;
R₂₂ is selected from hydrogen and C₁₋₆ alkyl;
R₂₃ is selected from —(CH₂)ₙNR₂₄ₐR_{24b} and —(CH₂)ₙOR₂₄ₐ;
R₂₄ₐ and R_{24b} are each independently selected from C₁₋₆ alkyl and phenyl, wherein the alkyl is substituted with 0 or 1 phenyl substituents;
optionally R₂₄ₐ and R_{24b} are taken together with the nitrogen to which they are attached to form a piperidine which is substituted with 0, 1 or 2 phenyl substituents;
n is a positive integer from 2 to 4;
and pharmaceutically acceptable salts thereof/
(c) compounds of formula (III): 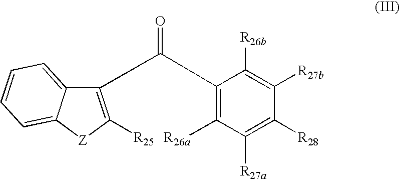
wherein R₂₅ is selected from hydrogen and C₁₋₆ alkyl;
R₂₆ₐ, R_{26b}, R₂₇ₐ, and R_{27b} are each independently selected from hydrogen, halogen and C₁₋₆ alkyl;
R₂₈ is selected from —O(CH₂)ₘNR₂₉ₐR_{29b} and —NH(CH₂)ₘNR₂₉ₐR_{29b};
R₂₉ₐ and R_{29b} are each independently selected from hydrogen and C₁₋₆ alkyl;
Z is O, S or NH;
m is a positive integer from 1 to 3;
and pharmaceutically acceptable salts thereof.
(d) compounds of formula (IV): 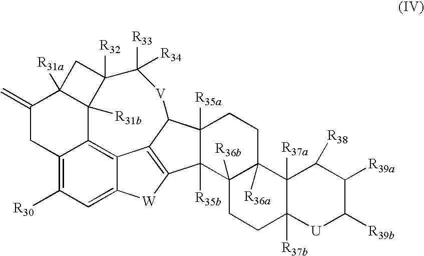
wherein R₃₀ is selected from hydrogen, C₁₋₆ alkyl and halogen;
R₃₁ₐ and R_{31b} are each independently selected from hydrogen, hydroxyl and C₁₋₆ alkyl;
R₃₂ is selected from hydrogen, hydroxyl and C₁₋₆ alkyl;
R₃₃ and R₃₄ are each independently selected from hydrogen and C₁₋₆ alkyl;
R₃₅ₐ and R_{35b} are each independently selected from hydrogen, hydroxyl and C₁₋₆ alkyl;
R₃₆ₐ and R_{36b} are each independently selected from hydrogen, hydroxyl and C₁₋₆ alkyl;
R₃₇ₐ and R_{37b} are each independently selected from hydrogen, hydroxyl and C₁₋₆ alkyl;
R₃₈ is selected from hydrogen, hydroxyl and C₁₋₆ alkyl;
optionally R₃₇ₐ and R₃₈ are taken together to form a three membered heterocycle of the formula: 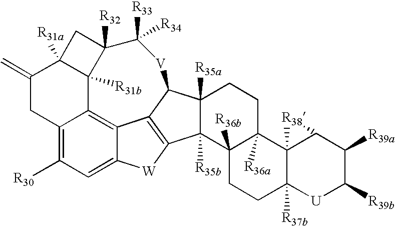
wherein R₃₈′ is O, S or NH;
R₃₉ₐ is selected from hydrogen, hydroxyl and C₁₋₆ alkyl;
R_{39b} is selected from hydrogen, hydroxyl, C₁₋₆ alkyl and C₂₋₆ alkenyl;
U, V and W are each independently selected from O, S, and NH;
and pharmaceutically acceptable salts thereof

22. (canceled)

23. (canceled)

24. A kit comprising: (i) a pharmaceutical composition comprising an autophagy inducing compound and (ii) instructions for administering the composition to a subject for the treatment of an autophagy associated disease.

25-40. (canceled)
